SEQUENCE LISTING (30 sequences)

SEQ ID NO: 1 - RNA, 61 nt, Hepatitis B virus
  source 1..61: mol_type = unassigned RNA
tgttcatgtcctactgttcaagcctccaagctgtgccttgggtggctttggggcatggaca

SEQ ID NO: 2 - DNA, 232 bp, Homo sapiens
  source 1..232: mol_type = unassigned DNA
ggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 3 - DNA, 1251 bp, Homo sapiens
  source 1..1251: mol_type = unassigned DNA
atggacgaagcggatcggcggctcctgcggcggtgccggctgcggctggtggaagagctgcaggtggaccagctctgggacgccctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataa

SEQ ID NO: 4 - DNA, 1251 bp, Homo sapiens
  source 1..1251: mol_type = unassigned DNA
ttatgatgttttaaagaaaagttttttccggaggaaattaaagcaaccaggcatctgtttataaatccctttcaccgaaacagcattagcgaccctaagcaggagggactgcaggtcttcagagtgagcccactgctcaaagatgtcgtccagggtctcaacgtaccaggagccactcttggggtccctccaggaaacaaaacctgggaaagtagagtaggacacaaagatgtcactgggtgtgggcaaactagatatggcgtccagctggtcgaaggtcctcaaaccttcctggaacggggtggcatctggctcggggttactgccaggggactcgtcttcaggggaagtggaggccacctcaaacccatggtctttctgctccccaccacaggcctggatgaaaaagagcttgggcttccctcccaggctggggcagctggtcccattgaagatgttcacaatcttctcgaccgacacagggcatccatctgtgccgtagacagcccctgggaactgcaggtggctggcctgacagccgtgagagagaatgaccaccacgcagcagtccagagcaccgtggtcctgctgcgccagctccagcaaagccagcaccattttcttggcagtcaggtcgcccttcacctccaccatgaaatgcagcgaggagaagcgacgccgcaacttctcacagtcgatgttggagccagtgcgggtgcggagcccggactcacggcagaagttcacattgttgataatgaggcagtggccacagggctccatgctcaggatgtaagccaaatctgcatttcccctcaaactctcaagagcaccgacatcaccaaatcctccagaaccaatgtccactggtctgggtgtttccggtctgagaacctctggtttgcgaatctctggtctgagcaccactggggtaaggttttctagggttggcttcgacaactttgctgcttgcctgttagttcgcagaaacgaagccagcatgtcctggcctgtgtcctctaagcaggagatgaacaaaggaagagcctgactccctcgagtctccagatctatgatcagctgcctggcctgatcccgccgagatccagagcctgcccgctggatgtcctcgatcatatggggcctgaacagctcgcggctcagcagggcgtcccagagctggtccacctgcagctcttccaccagccgcagccggcaccgccgcaggagccgccgatccgcttcgtccat

SEQ ID NO: 5 - DNA, 232 bp, Homo sapiens
  source 1..232: mol_type = unassigned DNA
cctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccactgacgggcaccggagcc

SEQ ID NO: 6 - DNA, 135 bp, Simian virus 40
  source 1..135: mol_type = unassigned DNA
acttgtttattgcagcttataatggttacaaataaagcaatagcatcacaaatttcacaaataaagcatttttttcactgcattctagttgtggtttgtccaaactcatcaatgtatcttatcatgtctggctct

SEQ ID NO: 7 - DNA, 135 bp, Simian virus 40
  source 1..135: mol_type = unassigned DNA
agagccagacatgataagatacattgatgagtttggacaaaccacaactagaatgcagtgaaaaaaatgctttatttgtgaaatttgtgatgctattgctttatttgtaaccattataagctgcaataaacaagt

SEQ ID NO: 8 - DNA, 1746 bp, synthetic construct
  misc_feature 1..1746: note = Synthetic sequence
  source 1..1746: mol_type = other DNA
atgtgttcatgtcctactgttcaagcctccaagctgtgccttgggtggctttggggcatggacaagagccagacatgataagatacattgatgagtttggacaaaccacaactagaatgcagtgaaaaaaatgctttatttgtgaaatttgtgatgctattgctttatttgtaaccattataagctgcaataaacaagtttatgatgttttaaagaaaagttttttccggaggaaattaaagcaaccaggcatctgtttataaatccctttcaccgaaacagcattagcgaccctaagcaggagggactgcaggtcttcagagtgagcccactgctcaaagatgtcgtccagggtctcaacgtaccaggagccactcttggggtccctccaggaaacaaaacctgggaaagtagagtaggacacaaagatgtcactgggtgtgggcaaactagatatggcgtccagctggtcgaaggtcctcaaaccttcctggaacggggtggcatctggctcggggttactgccaggggactcgtcttcaggggaagtggaggccacctcaaacccatggtctttctgctccccaccacaggcctggatgaaaaagagcttgggcttccctcccaggctggggcagctggtcccattgaagatgttcacaatcttctcgaccgacacagggcatccatctgtgccgtagacagcccctgggaactgcaggtggctggcctgacagccgtgagagagaatgaccaccacgcagcagtccagagcaccgtggtcctgctgcgccagctccagcaaagccagcaccattttcttggcagtcaggtcgcccttcacctccaccatgaaatgcagcgaggagaagcgacgccgcaacttctcacagtcgatgttggagccagtgcgggtgcggagcccggactcacggcagaagttcacattgttgataatgaggcagtggccacagggctccatgctcaggatgtaagccaaatctgcatttcccctcaaactctcaagagcaccgacatcaccaaatcctccagaaccaatgtccactggtctgggtgtttccggtctgagaacctctggtttgcgaatctctggtctgagcaccactggggtaaggttttctagggttggcttcgacaactttgctgcttgcctgttagttcgcagaaacgaagccagcatgtcctggcctgtgtcctctaagcaggagatgaacaaaggaagagcctgactccctcgagtctccagatctatgatcagctgcctggcctgatcccgccgagatccagagcctgcccgctggatgtcctcgatcatatggggcctgaacagctcgcggctcagcagggcgtcccagagctggtccacctgcagctcttccaccagccgcagccggcaccgccgcaggagccgccgatccgcttcgtccatcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccactgacgggcaccggagcctgttcatgtcctactgttcaagcctccaagctgtgccttgggtggctttggggcatggacataa

SEQ ID NO: 9 - DNA, 1191 bp, synthetic construct
  misc_feature 1..1191: note = Synthetic sequence
  source 1..1191: mol_type = other DNA
atgtgttcatgtcctactgttcaagcctccaagctgtgccttgggtggctttggggcatggacaagagccagacatgataagatacattgatgagtttggacaaaccacaactagaatgcagtgaaaaaaatgctttatttgtgaaatttgtgatgctattgctttatttgtaaccattataagctgcaataaacaagtttagggcaaggcggagccggaggcgatggcgtgctcggtcaggtgccacttctggttcttggcgtcgctgcggtcctcgcgggtcagcttgtgctggatgaagtgccagtcgggcatcttgcggggcacggacttggccttgtacacggtgtcgaactggcagcgcaagcggccaccgtccttcagcagcaggtacatgctcacgtcgcccttcaagatgccctgcttgggcacggggatgatcttctcgcaggagggctcccagttgtcggtcatcttcttcatcacggggccgtcggcggggaagttcacgccgtagaacttggactcgtggtacatgcagttctcctccacgctcacggtgatgtcggcgttgcagatgcacacggcgccgtcctcgaacaggaaggagcggtcccaggtgtagccggcggggcaggagttcttgaagtagtcgacgatgtcctgggggtactcggtgaacacgcggttgccgtacatgaaggcggcggacaagatgtcctcggcgaagggcaaggggccgccctccaccacgcacaggttgatggcctgcttgcccttgaaggggtagccgatgccctcgccggtgatcacgaacttgtggccgtccacgcagccctccatgcggtacttcatggtcatctccttggtcaggccgtgcttggactgggccatcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccactgacgggcaccggagcctgttcatgtcctactgttcaagcctccaagctgtgccttgggtggctttggggcatggacataa

SEQ ID NO: 10 - DNA, 12 bp, synthetic construct
  misc_feature 1..12: note = Synthetic sequence
  source 1..12: mol_type = other DNA
cctgcttttgct

SEQ ID NO: 11 - DNA, 13 bp, synthetic construct
  misc_feature 1..13: note = Synthetic sequence
  source 1..13: mol_type = other DNA
agtagaaacaagg

SEQ ID NO: 12 - DNA, 1292 bp, synthetic construct
  misc_feature 1..1292: note = Synthetic sequence
  source 1..1292: mol_type = other DNA
agtagaaacaagggtgttttttatcattatgatgttttaaagaaaagttttttccggaggaaattaaagcaaccaggcatctgtttataaatccctttcaccgaaacagcattagcgaccctaagcaggagggactgcaggtcttcagagtgagcccactgctcaaagatgtcgtccagggtctcaacgtaccaggagccactcttggggtccctccaggaaacaaaacctgggaaagtagagtaggacacaaagatgtcactgggtgtgggcaaactagatatggcgtccagctggtcgaaggtcctcaaaccttcctggaacggggtggcatctggctcggggttactgccaggggactcgtcttcaggggaagtggaggccacctcaaacccatggtctttctgctccccaccacaggcctggatgaaaaagagcttgggcttccctcccaggctggggcagctggtcccattgaagatgttcacaatcttctcgaccgacacagggcatccatctgtgccgtagacagcccctgggaactgcaggtggctggcctgacagccgtgagagagaatgaccaccacgcagcagtccagagcaccgtggtcctgctgcgccagctccagcaaagccagcaccattttcttggcagtcaggtcgcccttcacctccaccatgaaatgcagcgaggagaagcgacgccgcaacttctcacagtcgatgttggagccagtgcgggtgcggagcccggactcacggcagaagttcacattgttgataatgaggcagtggccacagggctccatgctcaggatgtaagccaaatctgcatttcccctcaaactctcaagagcaccgacatcaccaaatcctccagaaccaatgtccactggtctgggtgtttccggtctgagaacctctggtttgcgaatctctggtctgagcaccactggggtaaggttttctagggttggcttcgacaactttgctgcttgcctgttagttcgcagaaacgaagccagcatgtcctggcctgtgtcctctaagcaggagatgaacaaaggaagagcctgactccctcgagtctccagatctatgatcagctgcctggcctgatcccgccgagatccagagcctgcccgctggatgtcctcgatcatatggggcctgaacagctcgcggctcagcagggcgtcccagagctggtccacctgcagctcttccaccagccgcagccggcaccgccgcaggagccgccgatccgcttcgtccattcccctgcttttgct

SEQ ID NO: 13 - DNA, 13 bp, synthetic construct
  misc_feature 1..13: note = Synthetic sequence
  source 1..13: mol_type = other DNA
agtagaaacaagg

SEQ ID NO: 14 - DNA, 12 bp, synthetic construct
  misc_feature 1..12: note = Synthetic sequence
  source 1..12: mol_type = other DNA
cctgcttttgct

SEQ ID NO: 15 - RNA, 1292 nt, synthetic construct
  misc_feature 1..1292: note = Synthetic sequence
  source 1..1292: mol_type = other RNA
agtagaaacaagggtgttttttatcattatgatgttttaaagaaaagttttttccggaggaaattaaagcaaccaggcatctgtttataaatccctttcaccgaaacagcattagcgaccctaagcaggagggactgcaggtcttcagagtgagcccactgctcaaagatgtcgtccagggtctcaacgtaccaggagccactcttggggtccctccaggaaacaaaacctgggaaagtagagtaggacacaaagatgtcactgggtgtgggcaaactagatatggcgtccagctggtcgaaggtcctcaaaccttcctggaacggggtggcatctggctcggggttactgccaggggactcgtcttcaggggaagtggaggccacctcaaacccatggtctttctgctccccaccacaggcctggatgaaaaagagcttgggcttccctcccaggctggggcagctggtcccattgaagatgttcacaatcttctcgaccgacacagggcatccatctgtgccgtagacagcccctgggaactgcaggtggctggcctgacagccgtgagagagaatgaccaccacgcagcagtccagagcaccgtggtcctgctgcgccagctccagcaaagccagcaccattttcttggcagtcaggtcgcccttcacctccaccatgaaatgcagcgaggagaagcgacgccgcaacttctcacagtcgatgttggagccagtgcgggtgcggagcccggactcacggcagaagttcacattgttgataatgaggcagtggccacagggctccatgctcaggatgtaagccaaatctgcatttcccctcaaactctcaagagcaccgacatcaccaaatcctccagaaccaatgtccactggtctgggtgtttccggtctgagaacctctggtttgcgaatctctggtctgagcaccactggggtaaggttttctagggttggcttcgacaactttgctgcttgcctgttagttcgcagaaacgaagccagcatgtcctggcctgtgtcctctaagcaggagatgaacaaaggaagagcctgactccctcgagtctccagatctatgatcagctgcctggcctgatcccgccgagatccagagcctgcccgctggatgtcctcgatcatatggggcctgaacagctcgcggctcagcagggcgtcccagagctggtccacctgcagctcttccaccagccgcagccggcaccgccgcaggagccgccgatccgcttcgtccattcccctgcttttgct

SEQ ID NO: 16-17 - skipped (2 entries)

SEQ ID NO: 18 - DNA, 6734 bp, synthetic construct
  misc_feature 1..6734: note = Description of Artificial Sequence: Synthetic polynucleotide
  source 1..6734: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgtgttcatgtcctactgttcaagcctccaagctgtgccttgggtggctttggggcatggacaagagccagacatgataagatacattgatgagtttggacaaaccacaactagaatgcagtgaaaaaaatgctttatttgtgaaatttgtgatgctattgctttatttgtaaccattataagctgcaataaacaagtttatgatgttttaaagaaaagttttttccggaggaaattaaagcaaccaggcatctgtttataaatccctttcaccgaaacagcattagcgaccctaagcaggagggactgcaggtcttcagagtgagcccactgctcaaagatgtcgtccagggtctcaacgtaccaggagccactcttggggtccctccaggaaacaaaacctgggaaagtagagtaggacacaaagatgtcactgggtgtgggcaaactagatatggcgtccagctggtcgaaggtcctcaaaccttcctggaacggggtggcatctggctcggggttactgccaggggactcgtcttcaggggaagtggaggccacctcaaacccatggtctttctgctccccaccacaggcctggatgaaaaagagcttgggcttccctcccaggctggggcagctggtcccattgaagatgttcacaatcttctcgaccgacacagggcatccatctgtgccgtagacagcccctgggaactgcaggtggctggcctgacagccgtgagagagaatgaccaccacgcagcagtccagagcaccgtggtcctgctgcgccagctccagcaaagccagcaccattttcttggcagtcaggtcgcccttcacctccaccatgaaatgcagcgaggagaagcgacgccgcaacttctcacagtcgatgttggagccagtgcgggtgcggagcccggactcacggcagaagttcacattgttgataatgaggcagtggccacagggctccatgctcaggatgtaagccaaatctgcatttcccctcaaactctcaagagcaccgacatcaccaaatcctccagaaccaatgtccactggtctgggtgtttccggtctgagaacctctggtttgcgaatctctggtctgagcaccactggggtaaggttttctagggttggcttcgacaactttgctgcttgcctgttagttcgcagaaacgaagccagcatgtcctggcctgtgtcctctaagcaggagatgaacaaaggaagagcctgactccctcgagtctccagatctatgatcagctgcctggcctgatcccgccgagatccagagcctgcccgctggatgtcctcgatcatatggggcctgaacagctcgcggctcagcagggcgtcccagagctggtccacctgcagctcttccaccagccgcagccggcaccgccgcaggagccgccgatccgcttcgtccatcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccactgacgggcaccggagcctgttcatgtcctactgttcaagcctccaagctgtgccttgggtggctttggggcatggacataaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 19 - DNA, 6015 bp, synthetic construct
  misc_feature 1..6015: note = Description of Artificial Sequence: Synthetic polynucleotide
  source 1..6015: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttggggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatcaagtttgtacaaaaaagcaggctgccaccatgtgttcatgtcctactgttcaagcctccaagctgtgccttgggtggctttggggcatggacaagagccagacatgataagatacattgatgagtttggacaaaccacaactagaatgcagtgaaaaaaatgctttatttgtgaaatttgtgatgctattgctttatttgtaaccattataagctgcaataaacaagtttatgatgttttaaagaaaagttttttccggaggaaattaaagcaaccaggcatctgtttataaatccctttcaccgaaacagcattagcgaccctaagcaggagggactgcaggtcttcagagtgagcccactgctcaaagatgtcgtccagggtctcaacgtaccaggagccactcttggggtccctccaggaaacaaaacctgggaaagtagagtaggacacaaagatgtcactgggtgtgggcaaactagatatggcgtccagctggtcgaaggtcctcaaaccttcctggaacggggtggcatctggctcggggttactgccaggggactcgtcttcaggggaagtggaggccacctcaaacccatggtctttctgctccccaccacaggcctggatgaaaaagagcttgggcttccctcccaggctggggcagctggtcccattgaagatgttcacaatcttctcgaccgacacagggcatccatctgtgccgtagacagcccctgggaactgcaggtggctggcctgacagccgtgagagagaatgaccaccacgcagcagtccagagcaccgtggtcctgctgcgccagctccagcaaagccagcaccattttcttggcagtcaggtcgcccttcacctccaccatgaaatgcagcgaggagaagcgacgccgcaacttctcacagtcgatgttggagccagtgcgggtgcggagcccggactcacggcagaagttcacattgttgataatgaggcagtggccacagggctccatgctcaggatgtaagccaaatctgcatttcccctcaaactctcaagagcaccgacatcaccaaatcctccagaaccaatgtccactggtctgggtgtttccggtctgagaacctctggtttgcgaatctctggtctgagcaccactggggtaaggttttctagggttggcttcgacaactttgctgcttgcctgttagttcgcagaaacgaagccagcatgtcctggcctgtgtcctctaagcaggagatgaacaaaggaagagcctgactccctcgagtctccagatctatgatcagctgcctggcctgatcccgccgagatccagagcctgcccgctggatgtcctcgatcatatggggcctgaacagctcgcggctcagcagggcgtcccagagctggtccacctgcagctcttccaccagccgcagccggcaccgccgcaggagccgccgatccgcttcgtccatcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccactgacgggcaccggagcctgttcatgtcctactgttcaagcctccaagctgtgccttgggtggctttggggcatggacataaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 20 - DNA, 3177 bp, synthetic construct
  misc_feature 1..3177: note = Description of Artificial Sequence: Synthetic polynucleotide
  source 1..3177: mol_type = other DNA
taatacgactcactataggcatgaagacagtgtttagcaagattgtttttttgtcattctcctaagaagctattaaaatcacatggggatagcactactaaaattaattttacacattagggctcttccatataggcagctctccctagcattgttcactgtacactcgatcgtactccgcgtggcctcggtgaaaatgtggtggctctttcaagtcctccctaatgttacacactgattaaagattgttacaatgagctacctactgatcgcctgacacgatttcctgcacaggcttgagccatatactcatacatcgcatcttggccacgttttccacgggtttcaaaattaatctcaagttctacgcttaacgctttcgcctgttcccagttattaatatattcaacgctagaactcccctcagcgaagggaaggctgagcactacacgcgaagcaccatcaccgaaccttttgataaactcttccgttccgacttgctccatcaacggttcagtgagacttaaacctaactctttcttaatagtttcggcattatccacttttagtgcgagaaccttcgtcagtcctggatacgtcactttgaccacgcctccagcttttccagagagcgggttttcattatctacagagtatcccgcagcgtcgtatttattgtcggtactataaaaccctttccaatcatcgtcataatttccttgtgtaccagattttggcttttgtatacctttttgaatggaatctacataaccaggtttagtcccgtggtacgaagaaaagttttccatcacaaaagatttagaagaatcaacaacatcatcagcgcccatcttacctttcggtcacacccggacgaaacctagatgtgctgatgatcggctgcaacacggacgaaaccgtaagcagcctgcagaagatagacgagttactcgtgtcctgtcaacgacagtaattagttattaattatactgcgtgagtgcactaagcatgcagccgagtgacagccacacagattttaaagttcgtttagagaacagatctacaagagatcgaaagttggttggtttgttacctgggaaggtataaacctttaataaaaaaaaaaaaaaaaaaaaaaaaaaaaaatgaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 21 - DNA, 6079 bp, synthetic construct
  misc_feature 1..6079: note = Description of Artificial Sequence: Synthetic polynucleotide
  source 1..6079: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctcatgaagacagtgtttagcaagattgtttttttgtcattctcctaagaagctattaaaatcacatggggatagcactactaaaattaattttacacattagggctcttccatataggcagctctccctagcattgttcactgtacactcgatcgtactccgcgtggcctcggtgaaaatgtggtggctctttcaagtcctccctaatgttacacactgattaaagattgttacaatgagctacctactgatcgcctgacacgatttcctgcacaggcttgagccatatactcatacatcgcatcttggccacgttttccacgggtttcaaaattaatctcaagttctacgcttaacgctttcgcctgttcccagttattaatatattcaacgctagaactcccctcagcgaagggaaggctgagcactacacgcgaagcaccatcaccgaaccttttgataaactcttccgttccgacttgctccatcaacggttcagtgagacttaaacctaactctttcttaatagtttcggcattatccacttttagtgcgagaaccttcgtcagtcctggatacgtcactttgaccacgcctccagcttttccagagagcgggttttcattatctacagagtatcccgcagcgtcgtatttattgtcggtactataaaaccctttccaatcatcgtcataatttccttgtgtaccagattttggcttttgtatacctttttgaatggaatctacataaccaggtttagtcccgtggtacgaagaaaagttttccatcacaaaagatttagaagaatcaacaacatcatcagcgcccatcttacctttcggtcacacccggacgaaacctagatgtgctgatgatcggctgcaacacggacgaaaccgtaagcagcctgcagaagatagacgagttactcgtgtcctgtcaacgacagtaattagttattaattatactgcgtgagtgcactaagcatgcagccgagtgacagccacacagattttaaagttcgtttagagaacagatctacaagagatcgaaagttggttggtttgttacctgggaaggtataaacctttaatacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 22 - DNA, 29 bp, synthetic construct
  misc_feature 1..29: note = Synthetic sequence
  source 1..29: mol_type = other DNA
agtagaaacaagggtgttttttatcatta

SEQ ID NO: 23 - DNA, 18 bp, synthetic construct
  misc_feature 1..18: note = Synthetic sequence
  source 1..18: mol_type = other DNA
cattcccctgcttttgct

SEQ ID NO: 24 - DNA, 761 bp, synthetic construct
  misc_feature 1..761: note = Synthetic sequence
  source 1..761: mol_type = other DNA
agtagaaacaagggtgttttttatcattacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattcccctgcttttgct

SEQ ID NO: 25 - DNA, 265 bp, synthetic construct
  misc_feature 1..265: note = Description of Artificial Sequence: Synthetic polynucleotide
  source 1..265: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 26 - DNA, 229 bp, synthetic construct
  misc_feature 1..229: note = Description of Artificial Sequence: Synthetic polynucleotide
  source 1..229: mol_type = other DNA
caatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaacaatcttgctaaacactgtcttcatg

SEQ ID NO: 27 - DNA, 603 bp, synthetic construct
  misc_feature 1..603: note = Description of Artificial Sequence: Synthetic polynucleotide
  source 1..603: mol_type = other DNA
atgggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcagtaggtagctcattgtaa

SEQ ID NO: 28 - DNA, 1097 bp, synthetic construct
  misc_feature 1..1097: note = Description of Artificial Sequence: Synthetic polynucleotide
  source 1..1097: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatgggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcagtaggtagctcattgtaacaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaacaatcttgctaaacactgtcttcatg

SEQ ID NO: 29 - DNA, 603 bp, synthetic construct
  misc_feature 1..603: note = Description of Artificial Sequence: Synthetic polynucleotide
  source 1..603: mol_type = other DNA
ttacaatgagctacctactgatcgcctgacacgatttcctgcacaggcttgagccatatactcatacatcgcatcttggccacgttttccacgggtttcaaaattaatctcaagttctacgcttaacgctttcgcctgttcccagttattaatatattcaacgctagaactcccctcagcgaagggaaggctgagcactacacgcgaagcaccatcaccgaaccttttgataaactcttccgttccgacttgctccatcaacggttcagtgagacttaaacctaactctttcttaatagtttcggcattatccacttttagtgcgagaaccttcgtcagtcctggatacgtcactttgaccacgcctccagcttttccagagagcgggttttcattatctacagagtatcccgcagcgtcgtatttattgtcggtactataaaaccctttccaatcatcgtcataatttccttgtgtaccagattttggcttttgtatacctttttgaatggaatctacataaccaggtttagtcccgtggtacgaagaaaagttttccatcacaaaagatttagaagaatcaacaacatcatcagcgcccat

SEQ ID NO: 30 - DNA, 1097 bp, synthetic construct
  misc_feature 1..1097: note = Description of Artificial Sequence: Synthetic polynucleotide
  source 1..1097: mol_type = other DNA
catgaagacagtgtttagcaagattgtttttttgtcattctcctaagaagctattaaaatcacatggggatagcactactaaaattaattttacacattagggctcttccatataggcagctctccctagcattgttcactgtacactcgatcgtactccgcgtggcctcggtgaaaatgtggtggctctttcaagtcctccctaatgttacacactgattaaagattgttacaatgagctacctactgatcgcctgacacgatttcctgcacaggcttgagccatatactcatacatcgcatcttggccacgttttccacgggtttcaaaattaatctcaagttctacgcttaacgctttcgcctgttcccagttattaatatattcaacgctagaactcccctcagcgaagggaaggctgagcactacacgcgaagcaccatcaccgaaccttttgataaactcttccgttccgacttgctccatcaacggttcagtgagacttaaacctaactctttcttaatagtttcggcattatccacttttagtgcgagaaccttcgtcagtcctggatacgtcactttgaccacgcctccagcttttccagagagcgggttttcattatctacagagtatcccgcagcgtcgtatttattgtcggtactataaaaccctttccaatcatcgtcataatttccttgtgtaccagattttggcttttgtatacctttttgaatggaatctacataaccaggtttagtcccgtggtacgaagaaaagttttccatcacaaaagatttagaagaatcaacaacatcatcagcgcccatcttacctttcggtcacacccggacgaaacctagatgtgctgatgatcggctgcaacacggacgaaaccgtaagcagcctgcagaagatagacgagttactcgtgtcctgtcaacgacagtaattagttattaattatactgcgtgagtgcactaagcatgcagccgagtgacagccacacagattttaaagttcgtttagagaacagatctacaagagatcgaaagttggttggtttgttacctgggaaggtataaacctttaat

(19) United States
(12) Patent Application Publication
(10) Pub. No.: US 2026/0209279 A1
(43) Pub. Date: Jul. 23, 2026

(54) COMPOSITIONS AND METHODS FOR TREATING VIRAL INFECTIONS

(71) Applicant: G TECH BIO LLC, Los Angeles, CA (US)

(72) Inventor: Serhat Gumrukcu, Los Angeles, CA (US)

(73) Assignee: G TECH BIO LLC, LOS ANGELES, CA (US)

(21) Appl. No.: 19/201,010

(22) Filed: May 7, 2025

Related U.S. Application Data
(63) Continuation of application No. 17/005,733, filed on Aug. 28, 2020, now Pat. No. 12,319,714.
(60) Provisional application No. 62/896,460, filed on Sep. 5, 2019.
(60) Provisional application No. 62/968,387, filed on Jan. 31, 2020.
(60) Provisional application No. 62/976,491, filed on Feb. 14, 2020.
(60) Provisional application No. 62/985,597, filed on Mar. 5, 2020.

(51) Int. Cl. C07K 14/005; A61K 38/00; C12N 7/00; C12N 15/86

(52) U.S. Cl. CPC C07K 14/005; C12N 7/00; C12N 15/86; A61K 38/00; C12N 2730/10122; C12N 2730/10123; C12N 2730/10133; C12N 2750/14143

(57) ABSTRACT

The disclosure provides methods and compositions utilizing recombinant nucleic acid constructs or a replication incompetent virus-like particle encoding a chemokine, cytokine, or apoptosis inducing protein (e.g. Caspase 9 (Casp9)), or other toxins in a form which can only be transcribed in the presence of a viral polymerase. These methods can be adapted to target many viral infections and reduce or eliminate viral load, and provide a fundamentally different treatment for viral infections.

### CROSS-REFERENCE TO RELATED APPLICATIONS

This application is a Continuation of U.S. patent application Ser. No. 17/005,733, filed Aug. 28, 2020, which claims priority to U.S. Provisional Application No. 62/896,460, filed Sep. 5, 2019; U.S. Provisional Application No. 62/968,387, filed Jan. 31, 2020; U.S. Provisional Application No. 62/976,491, filed Feb. 14, 2020; and U.S. Provisional Application No. 62/985,597, filed Mar. 5, 2020, each of which are hereby incorporated by reference in their entirety.

### SEQUENCE LISTING

The instant application contains a Sequence Listing which has been submitted electronically in XML file format and is hereby incorporated by reference in its entirety. Said XML copy, created on Apr. 6, 2026, is named 12004_011626-US1_SL.xml and is 69,607 bytes in size.

### FIELD

The disclosure provides methods and compositions utilizing recombinant nucleic acid constructs or a replication incompetent virus-like particle encoding a chemokine, cytokine, or apoptosis inducing protein (e.g. Caspase 9 (Casp9)), in a form which will only be transcribed in the presence of a viral polymerase. These methods can be adapted to target many viral infections and reduce or eliminate viral load, and provide a fundamentally different treatment for viral infections.

### BACKGROUND

Viral infections are a challenging issue of global public health. Some progress has been made in treating certain viral infections, but many of these treatments are ineffective or cost-prohibitive for much of the world. For example, hepatitis B virus (HBV) infection, taken as a model for difficult to treat, chronic viral infections, remains a tremendous public health challenge. Since the introduction of HBV vaccination, the risk of transmission, including vertical mother-to-child transmission and horizontal transmission, has decreased dramatically. However, it is estimated that 248 million persons are still chronically infected by HBV worldwide. This imposes a high socioeconomic burden in the endemic regions and countries. The clinical outcomes of chronic hepatitis B (CHB) vary greatly, ranging from spontaneous resolution of hepatitis B to severe detrimental consequences, including the development of hepatic failure, cirrhosis, and hepatocellular carcinoma (HCC). Persistent or recurrent hepatic necroinflammation mediated by the host antiviral immunity is the major cause for the disease progression, eventually leading to advanced fibrosis and carcinogenesis of liver. The lifetime risk of severe adverse outcomes of CHB is as high as 15%-40%. Early diagnosis and timely antiviral treatment for those at risk are required to prevent the development of adverse clinical outcomes.

Thus, new, more effective and more widely available treatments are needed for treating viral infections, such as HBV. One of the challenging aspects in developing effective viral treatments are their complex life cycles, which are intimately connected to that of the host cell. For example HBV persistence in infected hepatocytes is due to the presence of covalently closed circular DNA (cccDNA), the template for the transcription of viral RNAs. Antiviral therapies with nucleoside analogues inhibit replication of HBV DNA in capsids present in the cytoplasm of infected cells, but do not reduce or destroy nuclear cccDNA. Despite some progress, antiviral drugs are currently only effective against a few viral diseases.

Thus, fundamentally different treatment approaches are needed to change the landscape for treating viral infections.

### SUMMARY

Provided herein is a recombinant nucleic acid sequence comprising: a negative strand nucleic acid molecule or a pgRNA nucleic acid molecule encoding a chemokine, a cytokine, an apoptosis inducing protein, or a combination thereof, flanked by a first and second viral transcription recognition signal, and further comprising a first promoter upstream (5′) of the first viral transcription recognition signal and a second promoter adjacent and 5′ to the negative strand nucleic acid molecule or pgRNA nucleic acid molecule encoding a chemokine, a cytokine, a apoptosis inducing protein, or combination thereof.

In certain embodiments, the negative strand nucleic acid molecule is negative sense RNA, negative sense DNA, single or double strand DNA that expresses a non-coding, negative sense RNA, or pgRNA, or any combination thereof.

In additional embodiments, the viral transcription recognition signal is selected from a virus selected from a negative strand virus, an RNA reverse transcribing virus, or a DNA reverse transcribing virus.

In additional embodiments, the recombinant nucleic acid further comprises a poly A tail downstream (3′) of the negative strand nucleic acid molecule or pgRNA nucleic acid molecule encoding a chemokine, a cytokine, an apoptosis inducing protein, or combination thereof.

In additional embodiments, the apoptosis inducing protein is selected from the group consisting of BAX, BID, BAK, BAD, caspase 2, caspase 8, caspase 9, caspase 10, caspase 11, caspase 12, cytochrome C, SMAC, and apoptosis-inducing factor, or combinations thereof.

In yet additional embodiments, the first promoter comprises a strong ubiquitous promoter or a liver-tissue-specific promoter, selected from the group consisting of TBG (Thyroxine Binding Globulin), albumin promoter and/or enhancing element, AFP (alpha-fetoprotein) promoter, AAT (Alpha-1-antitrypsin) promoter, ApoE (Apolipoprotein E) promoter or PEPCK (Phosphoenolpyruvate carboxykinase) promoter.

In yet additional embodiments, the second promoter comprises elongation factor 1alpha binding sequence (EFS).

In additional embodiments, the chemokine is selected from CCL1, CCL2, CCL3, CCL4, CCL5, CCL6, CCL7, CCL8, CCL9, CCL10, CCL 11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL8, CXCL9, CXCL10, CXCL 11, CXCL12, CXCL13, CXCL14, CXCL15, CXCL16, CXCL17, XCL1, XCL2, and CX3CL1.

In further embodiments, the cytokine is selected from the group consisting of IL-15, IL-2, IL-8, IL-10, IL-12, IL-6, IFN-α, IFN-β, IFN-γ, TNF-α, CD40L, Mig, and Crg-2.

In additional embodiments, the viral transcription recognition signal comprises epsilon recognition signal (SEQ ID NO:1).

In additional embodiments, the virus is Hepatitis B virus, Hepatitis D virus, ebola virus, Marburg virus, human parainfluenza virus 1, measles virus, mumps virus, human respiratory syncytial virus, vesicular stomatitis Indiana virus, rabies virus, bovine ephemeral fever virus, lymphocytic choriomeningitis virus, Bunyamwera virus, Hantaan virus, Nairobi sheep disease virus, sandfly fever Sicilian virus, influenza virus A, influenza virus C, Thogoto virus, mouse mammary tumor virus, murine leukemia virus, avian leukosis virus, Mason-Pfizer monkey virus, bovine leukemia virus, human immunodeficiency virus 1, human spumavirus, duck hepatitis B virus, and a combination thereof.

In additional embodiments, the nucleic acid molecule does not comprise sequences (coding or noncoding) for viral polymerase, reverse transcriptase, capsid, envelope, a packaging signal, or a translocation motif.

Provided herein is a vector comprising any of the recombinant nucleic acid sequences described herein.

In additional embodiments, the vector comprises a delivery vector or vehicle for delivery to a mammalian cell.

In additional embodiments, the vector or vehicle comprises a VLP, an adeno-associated virus (AAV), a liposome, a nanoparticle, a micelle, a polymeric vesicle, or a polymersome.

Provided herein is a pharmaceutical composition comprising any of the recombinant nucleic acid sequences described herein or any of the vectors described herein.

Provided herein is a replication incompetent virus-like particle (VLP) comprising:
an optional translocation motif (TLM) fused to a capsid protein from a hepatitis B viral capsid or a hepatitis D viral capsid; and
a negative strand nucleic acid molecule or a pgRNA nucleic acid molecule encoding a chemokine, a cytokine, a apoptosis inducing protein, or a combination thereof, flanked by a first and second viral transcription recognition signal, and further comprising a first promoter upstream (5′) of the first viral transcription recognition signal and a second promoter adjacent and 5′ to the negative strand nucleic acid molecule or pgRNA nucleic acid molecule encoding a chemokine, a cytokine, a apoptosis inducing protein, or a combination thereof.

Further provided herein is a replication incompetent virus-like particle (VLP) comprising:
a negative strand nucleic acid molecule or a pgRNA nucleic acid molecule encoding a chemokine, a cytokine, a apoptosis inducing protein, or a combination thereof, flanked by a first and second target viral transcription recognition signal, and further comprising a first promoter upstream (5′) of the first viral transcription recognition signal and a second promoter adjacent and 5′ to the negative strand nucleic acid molecule or pgRNA nucleic acid molecule encoding a chemokine, a cytokine, a apoptosis inducing protein, or a combination thereof, wherein the VLP exhibits tropism for the target virally infected cells.

In additional embodiments, the negative strand nucleic acid molecule is negative sense RNA, negative sense DNA, single or double strand DNA that expresses a non-coding, negative sense RNA, or pgRNA, or any combination thereof.

In additional embodiments, the replication incompetent virus-like particle (VLP) further comprises a poly A tail downstream (3′) of the negative strand nucleic acid molecule or pgRNA nucleic acid molecule encoding a chemokine, a cytokine, a apoptosis inducing protein, or combination thereof.

In additional embodiments, the first promoter comprises a strong ubiquitous promoter or a liver-tissue-specific promoter selected from the group consisting of TBG (Thyroxine Binding Globulin), albumin promoter and/or enhancing element, AFP (alpha-fetoprotein) promoter, AAT (Alpha-1-antitrypsin) promoter, ApoE (Apolipoprotein E) promoter or PEPCK (Phosphoenolpyruvate carboxykinase) promoter.

In additional embodiments, the second promoter comprises elongation factor 1alpha binding sequence (EFS).

In additional embodiments, the viral transcription recognition signal is selected from a virus selected from a negative strand virus, an RNA reverse transcribing virus, or a DNA reverse transcribing virus.

In additional embodiments, the viral transcription recognition signal comprises the epsilon recognition signal (SEQ ID NO:1).

In additional embodiments, the apoptosis inducing protein is selected from the group consisting of BAX, BID, BAK, BAD, caspase 2, caspase 8, caspase 9, caspase 10, caspase 11, caspase 12, cytochrome C, SMAC, and apoptosis-inducing factor.

In additional embodiments, the chemokine is selected from CCL1, CCL2, CCL3, CCL4, CCL5, CCL6, CCL7, CCL8, CCL9, CCL10, CCL 11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL8, CXCL9, CXCL10, CXCL 11, CXCL12, CXCL13, CXCL14, CXCL15, CXCL16, CXCL17, XCL1, XCL2, and CX3CL1.

In additional embodiments, the cytokine is selected from the group consisting of IL-15, IL-2, IL-8, IL-10, IL-12, IL-6, IFN-α, IFN-β, IFN-γ, TNF-α, CD40L, Mig, and Crg-2.

In additional embodiments, the virus is Hepatitis B virus, Hepatitis D virus, ebola virus, Marburg virus, human parainfluenza virus 1, measles virus, mumps virus, human respiratory syncytial virus, vesicular stomatitis Indiana virus, rabies virus, bovine ephemeral fever virus, lymphocytic choriomeningitis virus, Bunyamwera virus, Hantaan virus, Nairobi sheep disease virus, sandfly fever Sicilian virus, influenza virus A, influenza virus C, Thogoto virus, mouse mammary tumor virus, murine leukemia virus, avian leukosis virus, Mason-Pfizer monkey virus, bovine leukemia virus, human immunodeficiency virus 1, human spumavirus, duck hepatitis B virus, and a combination thereof.

In additional embodiments, the nucleic acid molecule does not comprise sequences (coding or noncoding) for viral capsid, polymerase, reverse transcriptase, envelope, packaging signal, or translocation motifs.

Further provided is a pharmaceutical composition comprising any of the incompetent virus-like particles described herein.

Further provided is a method of treating a viral infection in a subject in need thereof, the method comprising administering to the subject a recombinant nucleic acid molecule described herein, or the vector described herein, or pharmaceutical compositions described herein, or the replication incompetent virus-like particle described herein.

In additional embodiments, the viral infection comprises an infection from a virus classified under Baltimore Classification IV, V, VI, or VII.

In additional embodiments, the viral infection comprises an infection from a virus selected from the group consisting of Hepatitis B virus, Hepatitis D virus, ebola virus, Marburg virus, human parainfluenza virus 1, measles virus, mumps virus, human respiratory syncytial virus, vesicular stomatitis Indiana virus, rabies virus, bovine ephemeral fever virus, lymphocytic choriomeningitis virus, Bunyamwera virus, Hantaan virus, Nairobi sheep disease virus, sandfly fever Sicilian virus, influenza virus A, influenza virus C, Thogoto virus, mouse mammary tumor virus, murine leukemia virus, avian leukosis virus, Mason-Pfizer monkey virus, bovine leukemia virus, human immunodeficiency virus 1, human spumavirus, duck hepatitis B virus, and a combination thereof.

In additional embodiments, the viral infection comprises an infection from a virus selected from the group consisting of influenza A, B, C, and/or any coronavirus, and/or any viruses from Baltimore Classification Group IV.

In additional embodiments, the viral infection comprises an infection from a virus selected from the group consisting of filovirus, paramyxovirus, morbillivirus, rubulavirus, pneumovirus, vesiculovirus, lyssavrius, ephemerovirus, arenavirus, bunyavirus, hantavirus, nairovirus, phlebovirus, Orthohepadnavirus, Avihepadnavirus, Mammalian type B retroviruses, Mammalian type C retroviruses, Avian type C retroviruses, Type D retroviruses, BLV-HTLV retroviruses, Lentivirus, Spumavirus, and a combination thereof.

In some embodiments, the viral infection comprises an infection from a coronavirus, such as the virus referred to as COVID-19, SARS, or MERS.

In additional embodiments, the nucleic acid molecule, vector, pharmaceutical composition, or replication incompetent virus-like particle upon administration enters a liver cell.

In additional embodiments, the nucleic acid molecule, vector, pharmaceutical composition, or replication incompetent virus-like particle upon administration delivers the nucleic acid molecule to a liver cell and the nucleic molecule is expressed in the liver cell.

In additional embodiments, the subject has an acute, chronic, or latent viral infection.

In additional embodiments, administration induces an immune response against a cell infected with the virus causing the viral infection.

In additional embodiments, administration induces apoptosis in cells infected with the virus causing the viral infection.

Further provided is a method of inducing an immune response against a cell infected with a virus, in a subject in need thereof, the method comprising contacting the subject with any of the recombinant nucleic acid molecules described herein, or the vectors described herein, or the pharmaceutical compositions described herein, or the replication incompetent virus-like particles described herein.

Further provided is a method of inducing an apoptotic response against a cell infected with a virus, in a subject in need thereof, the method comprising contacting the subject with any of the recombinant nucleic acid molecules described herein, or the vectors described herein, or the pharmaceutical compositions described herein, or the replication incompetent virus-like particles described herein.

In additional embodiments, the virus is HBV, HDV, hepatitis A virus (HAV), hepatitis C virus (HCV), or any combination thereof.

Further provided is a method of treating hepatitis B in a subject, the method comprising administering to the subject a recombinant nucleic acid molecule described herein, or a vector described herein, or the pharmaceutical compositions described herein, or the replication incompetent virus-like particle described herein.

In additional embodiments, the method further comprises administering at least one antiviral agent, HBV polymerase inhibitor, interferon, TLR modulators such as TLR-7 agonists or TLR-9 agonists, therapeutic vaccines, immune activator of certain cellular viral RNA sensors, viral entry inhibitor, viral maturation inhibitor, distinct capsid assembly modulator, antiviral compounds of distinct or unknown mechanism, and any combination thereof.

In additional embodiments, the method further comprises administering at least one antiviral agent 3TC, FTC, L-FMAU, interferon, adefovir dipivoxil, entecavir, telbivudine (L-dT), valtorcitabine (3′-valinyl L-dC), .beta.-D-dioxolanyl-guanine (DXG), .beta.-D-dioxolanyl-2,6-diaminopurine (DAPD), .beta.-D-dioxolanyl-6-chloropurine (ACP), famciclovir, penciclovir, lobucavir, ganciclovir, ribavirin, and any combination thereof.

In additional embodiments, the treatment is administered periodically, including once every week, once every 2 weeks, once every 3 weeks, once a month, to once every two months, to once every 3 months, to once every 4 months, to once every 5 months, to once every 6 months, or once every 7 months, or once every 8 months, or once every 9 months, or once every 10 months, or every 11 months, or once annually as a maintenance treatment, or for as long as the patient requires to achieve stable or undetectable disease.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A and FIG. 1B include a plasmid map and recombinant construct schematic showing features of the AAV8-HBV-DRS2 construct (6005 bp).

FIG. 2A, FIG. 2B, and FIG. 2C illustrate a non-limiting plasmid map, a graph showing viable cell data of test and control constructs in HepG2 (wild-type) and HepAD38 cells, along with the numeric data for experiment 1.

FIG. 3A and FIG. 3B illustrate a non-limiting plasmid map and chemical structure of a Caspase-9 inhibitor and a graph showing viable cell data of test and control constructs in HepG2 (wild-type) cells for experiment 2.

FIG. 4A, FIG. 4B, and FIG. 4C illustrate a non-limiting plasmid map and chemical structure of the Caspase-9 inhibitor (FIG. 4A), a graph showing viable cell data of test and control constructs in HepAD38 cells (HBV model cells) (FIG. 4B), along with the numeric data for experiment 2 (FIG. 4C).

FIG. 5A and FIG. 5B show a plasmid map (FIG. 5A) and comparative graph (FIG. 5B) showing the results for test and control constructs in HepG2 vs HepAD38 cells for experiment 2.

FIG. 6A, FIG. 6B, and FIG. 6C show a plasmid map for test construct AAV-HBV-DRS2 (TBG>HBV-rcCasp9) and chemical structure of the Caspase-9 inhibitor (FIG. 6A), and a comparative graph showing viable cell data of test and control constructs in HepG2 vs. HepAD38 cells (FIG. 6B), along with the numeric data for experiment 3 (FIG. 6C).

FIG. 7 is a plasmid map and construct schematic showing features of the AAV8-HBV-DRS1 construct and sequences (SEQ ID. NO: 18).

FIG. 8 is a plasmid map and construct schematic showing features of the AAV8-HBV-DRS2 construct and sequences (SEQ ID. NO: 19).

FIG. 9 is a schematic illustrating the in vivo testing utilizing model mice to evaluate distribution, efficacy, specificity/functionality, and safety of the targeted recombinant constructs.

FIG. 10A and FIG. 10B are a schematic and a graph showing how the “influenza hijack/suicide vectors” hijack the viral machinery to induce apoptosis in infected cells (FIG. 10A), and in vitro results showing that the test constructs delivered in trans to engage with influenza polymerase hijacks the viral machinery to induce cell death in influenza infected cells 40% more rapidly than untreated infected cells (FIG. 10B).

FIG. 11A, FIG. 11B, and FIG. 11C are images, graphs and a schematic showing results using the HBV hijack constructs in mouse models.

FIG. 12A and FIG. 12B are schematics showing the overview of design for a coronavirus “hijack-RNA” construct.

FIG. 13 illustrates a schematic showing the mechanism of action of SARS-CoV-2 hijack RNA in SARS-CoV-2-infected cells.

FIG. 14 illustrates a non-limiting plasmid map and construct schematic showing features and sequence of the Cov-2 Hijack DTA in vitro transcription vector (SEQ ID. NO: 20).

FIG. 15 illustrates a non-limiting plasmid map and construct schematic showing features and sequence of the SARS-CoV-2 hijack RNA construct (SEQ ID. NO: 21).

FIG. 16A and FIG. 16B are comparative graphs showing viability of SARS-CoV-2-infected or uninfected cells following treatment with test or control constructs.

### DETAILED DESCRIPTION

In certain embodiments, methods and compositions utilizing recombinant nucleic acid constructs or a replication incompetent virus-like particle encoding a chemokine, cytokine, or apoptosis inducing protein (e.g. Caspase 9 (Casp9) and others as provided herein), which will only be transcribed in the presence of a viral polymerase are provided. In certain embodiments, constructs carry sequences encoding for Casp9, which will result in killing of virally infected cells. These methods can be adapted to target many viral infections and reduce or eliminate viral load, and provide a fundamentally different treatment for viral infections.

### Utilizing Viral Machinery to Destroy Virally Infected Cells

While not wishing to be bound by theory, the present methods and compositions are based at least in part on utilizing the viral machinery present typically in the cytoplasm of an infected cell. In certain embodiments, directing or injecting a recombinant nucleic acid construct or a replication incompetent virus-like particle (VLP) into a virally infected cell, wherein the construct or VLP comprises a single stranded RNA nucleic acid construct containing a sequence coding for a chemokine, cytokine, or apoptosis inducing protein, e.g., Casp 9 (and its promoter), flanked by hepatitis B epsilon signal binding sequences, to form a construct that will only be transcribed when recognized by the hepatitis B reverse transcriptase, will engage the epsilon sequence to transcribe the segment and result in translation of the coding sequence for Casp 9, which will trigger apoptosis of the hepatitis B infected cell. These constructs will effectively function as “viral specific suicide constructs” which will otherwise be degraded in non-virally infected cells.

Thus, in certain embodiments, the present compositions and methods encompass recombinant nucleic acid constructs and replication incompetent virus-like particles designed to target Hepatitis B infected cells. HBV is from Baltimore Group VII, which include double-stranded DNA viruses that replicate through a single-stranded RNA intermediate. This small group of viruses, exemplified by the Hepatitis B virus, have a double-stranded, gapped genome that is subsequently filled in to form a covalently closed circle (cccDNA) that serves as a template for production of viral mRNAs and a subgenomic RNA. The pregenome RNA serves as template for the viral reverse transcriptase for production of the DNA genome. This viral polymerase is what recognizes the flanking epsilon sequences in the recombinant nucleic acid constructs and replication incompetent virus-like particles described herein, and results in production of the toxic agent. Thus, only the virally infected cells will be killed by the production of the chemokine, cytokine, or apoptosis inducing protein (e.g. Caspase 9 (Casp9)).

In some embodiments, the chemokine, cytokine, or apoptosis inducing protein is flanked by the viral 5′ UTR and the viral 3′ UTR, such that the nucleic acid can be represented by the formula X—Y—Z, wherein X is the viral 5′ UTR, Y is the protein of interest, such as a chemokine, cytokine, or apoptosis inducing protein (e.g. Caspase 9 or others as provided for herein, or diphtheria toxin A fragment), and Z is the viral 3′ UTR. There may be intervening sequences between the 5′ UTR and the protein of interest or the 3′ UTR and the protein of interest. When the transcript is recognized by the cell it will produce a 5′-3′ reverse complement encoding the protein of interest. This can be delivered as well in an AAV expression vector or other appropriate viral vector.

In some embodiments, the 5′-UTR is the 5′ Leading Sequence of a coronavirus such as the virus that is referred to as COVID-19 (or COVD-19), SARS, or MERS. In some embodiments, the 5′-UTR comprises the sequence, or complement thereof, of:

(SEQ ID NO: 25)
ATTAAAGGTTTATACCTTCCCAGGTAACAAACCAACCAACTTTCGATCT
CTTGTAGATCTGTTCTCTAAACGAACTTTAAAATCTGTGTGGCTGTCAC
TCGGCTGCATGCTTAGTGCACTCACGCAGTATAATTAATAACTAATTAC
TGTCGTTGACAGGACACGAGTAACTCGTCTATCTTCTGCAGGCTGCTTA
CGGTTTCGTCCGTGTTGCAGCCGATCATCAGCACATCTAGGTTTCGTCC
GGGTGTGACCGAAAGGTAAG

In some embodiments, the 3′UTR comprises the sequence, or complement thereof, of:

(SEQ ID NO: 26)
CAATCTTTAATCAGTGTGTAACATTAGGGAGGACTTGAAAGAGCCACCA
CATTTTCACCGAGGCCACGCGGAGTACGATCGAGTGTACAGTGAACAAT
GCTAGGGAGAGCTGCCTATATGGAAGAGCCCTAATGTGTAAAATTAATT
TTAGTAGTGCTATCCCCATGTGATTTTAATAGCTTCTTAGGAGAATGAC
AAAAAAACAATCTTGCTAAACACTGTCTTCATG

In some embodiments, the nucleic acid sequence encoding diphtheria toxin A fragment comprises (or the complement thereof):

(SEQ ID NO: 27)
ATGGGCGCTGATGATGTTGTTGATTCTTCTAAATCTTTTGTGATGGAAA
ACTTTTCTTCGTACCACGGGACTAAACCTGGTTATGTAGATTCCATTCA
AAAAGGTATACAAAAGCCAAAATCTGGTACACAAGGAAATTATGACGAT
GATTGGAAAGGGTTTTATAGTACCGACAATAAATACGACGCTGCGGGAT
ACTCTGTAGATAATGAAAACCCGCTCTCTGGAAAAGCTGGAGGCGTGGT
CAAAGTGACGTATCCAGGACTGACGAAGGTTCTCGCACTAAAAGTGGAT
AATGCCGAAACTATTAAGAAAGAGTTAGGTTTAAGTCTCACTGAACCGT
TGATGGAGCAAGTCGGAACGGAAGAGTTTATCAAAAGGTTCGGTGATGG
TGCTTCGCGTGTAGTGCTCAGCCTTCCCTTCGCTGAGGGGAGTTCTAGC
GTTGAATATATTAATAACTGGGAACAGGCGAAAGCGTTAAGCGTAGAAC
TTGAGATTAATTTTGAAACCCGTGGAAAACGTGGCCAAGATGCGATGTA
TGAGTATATGGCTCAAGCCTGTGCAGGAAATCGTGTCAGGCGATCAGTA
GGTAGCTCATTGTAA

In some embodiments, a composition comprising the sequence of:

(SEQ ID NO: 28)
ATTAAAGGTTTATACCTTCCCAGGTAACAAACCAACCAACTTTCGATCT
CTTGTAGATCTGTTCTCTAAACGAACTTTAAAATCTGTGTGGCTGTCAC
TCGGCTGCATGCTTAGTGCACTCACGCAGTATAATTAATAACTAATTAC
TGTCGTTGACAGGACACGAGTAACTCGTCTATCTTCTGCAGGCTGCTTA
CGGTTTCGTCCGTGTTGCAGCCGATCATCAGCACATCTAGGTTTCGTCC
GGGTGTGACCGAAAGGTAAGATGGGCGCTGATGATGTTGTTGATTCTTC
TAAATCTTTTGTGATGGAAAACTTTTCTTCGTACCACGGGACTAAACCT
GGTTATGTAGATTCCATTCAAAAAGGTATACAAAAGCCAAAATCTGGTA
CACAAGGAAATTATGACGATGATTGGAAAGGGTTTTATAGTACCGACAA
TAAATACGACGCTGCGGGATACTCTGTAGATAATGAAAACCCGCTCTCT
GGAAAAGCTGGAGGCGTGGTCAAAGTGACGTATCCAGGACTGACGAAGG
TTCTCGCACTAAAAGTGGATAATGCCGAAACTATTAAGAAAGAGTTAGG
TTTAAGTCTCACTGAACCGTTGATGGAGCAAGTCGGAACGGAAGAGTTT
ATCAAAAGGTTCGGTGATGGTGCTTCGCGTGTAGTGCTCAGCCTTCCCT
TCGCTGAGGGGAGTTCTAGCGTTGAATATATTAATAACTGGGAACAGGC
GAAAGCGTTAAGCGTAGAACTTGAGATTAATTTTGAAACCCGTGGAAAA
CGTGGCCAAGATGCGATGTATGAGTATATGGCTCAAGCCTGTGCAGGAA
ATCGTGTCAGGCGATCAGTAGGTAGCTCATTGTAACAATCTTTAATCAG
TGTGTAACATTAGGGAGGACTTGAAAGAGCCACCACATTTTCACCGAGG
CCACGCGGAGTACGATCGAGTGTACAGTGAACAATGCTAGGGAGAGCTG
CCTATATGGAAGAGCCCTAATGTGTAAAATTAATTTTAGTAGTGCTATC
CCCATGTGATTTTAATAGCTTCTTAGGAGAATGACAAAAAAACAATCTT
GCTAAACACTGTCTTCATG

is provided, which encodes the 5′UTR, the 3′UTR and the protein of interest, which can be any sequence as provided herein. In the example above, the sequence encodes a diphtheria toxin A fragment.

In some embodiments, the nucleic acid sequence encoding diphtheria toxin A fragment is provided as the reverse complement, which can comprise the sequence of:

(SEQ ID NO: 29)
TTACAATGAGCTACCTACTGATCGCCTGACACGATTTCCTGCACAGGCT
TGAGCCATATACTCATACATCGCATCTTGGCCACGTTTTCCACGGGTTT
CAAAATTAATCTCAAGTTCTACGCTTAACGCTTTCGCCTGTTCCCAGTT
ATTAATATATTCAACGCTAGAACTCCCCTCAGCGAAGGGAAGGCTGAGC
ACTACACGCGAAGCACCATCACCGAACCTTTTGATAAACTCTTCCGTTC
CGACTTGCTCCATCAACGGTTCAGTGAGACTTAAACCTAACTCTTTCTT
AATAGTTTCGGCATTATCCACTTTTAGTGCGAGAACCTTCGTCAGTCCT
GGATACGTCACTTTGACCACGCCTCCAGCTTTTCCAGAGAGCGGGTTTT
CATTATCTACAGAGTATCCCGCAGCGTCGTATTTATTGTCGGTACTATA
AAACCCTTTCCAATCATCGTCATAATTTCCTTGTGTACCAGATTTTGGC
TTTTGTATACCTTTTTGAATGGAATCTACATAACCAGGTTTAGTCCCGT
GGTACGAAGAAAAGTTTTCCATCACAAAAGATTTAGAAGAATCAACAAC
ATCATCAGCGCCCAT

In some embodiments, sequence is provided as the reverse complement, which can comprise the sequence of:

(SEQ ID NO: 30)
CATGAAGACAGTGTTTAGCAAGATTGTTTTTTTGTCATTCTCCTAAGAA
GCTATTAAAATCACATGGGGATAGCACTACTAAAATTAATTTTACACAT
TAGGGCTCTTCCATATAGGCAGCTCTCCCTAGCATTGTTCACTGTACAC
TCGATCGTACTCCGCGTGGCCTCGGTGAAAATGTGGTGGCTCTTTCAAG
TCCTCCCTAATGTTACACACTGATTAAAGATTGTTACAATGAGCTACCT
ACTGATCGCCTGACACGATTTCCTGCACAGGCTTGAGCCATATACTCAT
ACATCGCATCTTGGCCACGTTTTCCACGGGTTTCAAAATTAATCTCAAG
TTCTACGCTTAACGCTTTCGCCTGTTCCCAGTTATTAATATATTCAACG
CTAGAACTCCCCTCAGCGAAGGGAAGGCTGAGCACTACACGCGAAGCAC
CATCACCGAACCTTTTGATAAACTCTTCCGTTCCGACTTGCTCCATCAA
CGGTTCAGTGAGACTTAAACCTAACTCTTTCTTAATAGTTTCGGCATTA
TCCACTTTTAGTGCGAGAACCTTCGTCAGTCCTGGATACGTCACTTTGA
CCACGCCTCCAGCTTTTCCAGAGAGCGGGTTTTCATTATCTACAGAGTA
TCCCGCAGCGTCGTATTTATTGTCGGTACTATAAAACCCTTTCCAATCA
TCGTCATAATTTCCTTGTGTACCAGATTTTGGCTTTTGTATACCTTTTT
GAATGGAATCTACATAACCAGGTTTAGTCCCGTGGTACGAAGAAAAGTT
TTCCATCACAAAAGATTTAGAAGAATCAACAACATCATCAGCGCCCATC
TTACCTTTCGGTCACACCCGGACGAAACCTAGATGTGCTGATGATCGGC
TGCAACACGGACGAAACCGTAAGCAGCCTGCAGAAGATAGACGAGTTAC
TCGTGTCCTGTCAACGACAGTAATTAGTTATTAATTATACTGCGTGAGT
GCACTAAGCATGCAGCCGAGTGACAGCCACACAGATTTTAAAGTTCGTT
TAGAGAACAGATCTACAAGAGATCGAAAGTTGGTTGGTTTGTTACCTGG
GAAGGTATAAACCTTTAAT

Although the sequences provided herein (above and below) are represented as DNA sequences, the corresponding RNA sequences are also provided.

The terms “nucleic acid sequence” and “nucleic acid molecule”, as used herein, can be used interchangeably.

In some embodiments, a recombinant nucleic acid sequence is provided that comprises a negative strand nucleic acid molecule or a pgRNA nucleic acid molecule encoding a chemokine, a cytokine, an apoptosis inducing protein, or a combination thereof, flanked by a first and second viral transcription recognition signal, and further comprising a first promoter upstream (5′) of the first viral transcription recognition signal and a second promoter adjacent and 5′ to the negative strand nucleic acid molecule or pgRNA nucleic acid molecule encoding a chemokine, a cytokine, a apoptosis inducing protein, diphtheria toxin A (or fragment thereof), or any combination thereof. Non-limiting examples of chemokines, cytokines, and apoptosis inducing proteins are provided herein. The actual protein that is flanked by the first and second viral transcription recognition signal can be any suitable protein. This encoded protein can also be replaced with any protein of interest, not just that encode for a chemokine, a cytokine, a apoptosis inducing protein, or a diphtheria toxin A (or fragment thereof).

As provided herein the negative strand nucleic acid molecule can be a negative sense RNA, negative sense DNA, single or double strand DNA that expresses a non-coding, negative sense RNA, or pgRNA, or any combination thereof. In some embodiments, the negative strand nucleic acid molecule is a negative sense RNA. In some embodiments, the negative strand nucleic acid molecule is a negative sense DNA. In some embodiments, the negative strand nucleic acid molecule is a single or double strand DNA that expresses a non-coding, negative sense RNA. In some embodiments, the negative strand nucleic acid molecule is a pgRNA.

As provided herein, in some embodiments, the viral transcription recognition signal is derived from or based on a virus that is, for example, a negative strand virus, an RNA reverse transcribing virus, or a DNA reverse transcribing virus. In some embodiments, the viral transcription recognition signal is a negative strand virus viral transcription recognition signal. In some embodiments, the viral transcription recognition signal is a RNA reverse transcribing virus viral transcription recognition signal. In some embodiments, the viral transcription recognition signal is a DNA reverse transcribing virus viral transcription recognition signal.

In some embodiments, the recombinant nucleic acid sequence comprises a poly A tail downstream (3′) of the negative strand nucleic acid molecule or pgRNA nucleic acid molecule that also encodes the protein of interest, such as a toxin, chemokine, a cytokine, an apoptosis inducing protein, or combination thereof.

The apoptosis inducing protein can be any protein that is capable of inducing apoptosis based on its expression in a cell. Examples include, but are not limited to, BAX, BID, BAK, BAD, caspase 2, caspase 8, caspase 9, caspase 10, caspase 11, caspase 12, cytochrome C, SMAC, and apoptosis-inducing factor, or combinations thereof. Thus, these proteins can be expressed as the protein of interest in the construct that is administered to an infected individual or contacted with an infected cell to induce the cell death through the expression of these proteins of interest.

In some embodiments, the protein of interest is not a viral protein.

Examples of chemokines that can be used or encoded for by the present embodiments, include, but are not limited to, CCL1, CCL2, CCL3, CCL4, CCL5, CCL6, CCL7, CCL8, CCL9, CCL10, CCL 11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL8, CXCL9, CXCL10, CXCL 11, CXCL12, CXCL13, CXCL14, CXCL15, CXCL16, CXCL17, XCL1, XCL2, and/or CX3CL1. In some embodiments, the cytokine is selected from the group consisting of IL-15, IL-2, IL-8, IL-10, IL-12, IL-6, IFN-α, IFN-β, IFN-γ, TNF-α, CD40L, Mig, and Crg-2.

As provided herein, in some embodiments, the recombinant nucleic acid sequence can further comprise a promoter that directs the expression of the nucleic acid sequences in the cell. In some embodiments, the promoter is a constitutive promoter. In some embodiments, the promoter is a tissue specific promoter. In some embodiments, the promoter is a liver-tissue-specific promoter. Examples of liver-tissue-specific promoters include, but are not limited to, TBG (Thyroxine Binding Globulin), albumin promoter and/or enhancing element, AFP (alpha-fetoprotein) promoter, AAT (Alpha-1-antitrypsin) promoter, ApoE (Apolipoprotein E) promoter or PEPCK (Phosphoenolpyruvate carboxykinase) promoter. The promoters can be used as the first promoter as referenced herein.

In some embodiments, the recombinant nucleic acid sequence comprises a second promoter. A non-limiting example of such a second promoter is elongation factor 1alpha binding sequence (EFS).

In some embodiments, the viral transcription recognition signal (sequence) comprises epsilon recognition signal (SEQ ID NO:1) or a corona virus recognition sequence (such as those found in SEQ ID: 25, SEQ ID: 26, SEQ ID: 28, or SEQ ID: 30). Other viral transcription recognition signal sequences can also be used and be substituted for one that is specific for the virus or viral infection to be treated.

As provided herein, the present embodiments can be used to treat viral infections. And in some embodiments, the compositions and methods can be used to specifically kill virally infected cells. The benefit of specifically killing virally infected cells is that it can lead to the destruction of only of the cells that harbor the virus. Examples of viruses that can be used for the viral transcription recognition signal include, but are not limited to, corona virus (e.g. COVID-19, SARS, MERS), Hepatitis B virus, Hepatitis D virus, ebola virus, Marburg virus, human parainfluenza virus 1, measles virus, mumps virus, human respiratory syncytial virus, vesicular stomatitis Indiana virus, rabies virus, bovine ephemeral fever virus, lymphocytic choriomeningitis virus, Bunyamwera virus, Hantaan virus, Nairobi sheep disease virus, sandfly fever Sicilian virus, influenza virus A, influenza virus C, Thogoto virus, mouse mammary tumor virus, murine leukemia virus, avian leukosis virus, Mason-Pfizer monkey virus, bovine leukemia virus, human immunodeficiency virus 1, human spumavirus, duck hepatitis B virus, coronavirus, and a combination thereof. Without being bound to any particular theory, the choice of the viral transcription recognition signal sequence can be used to determine which type of viral infection is treated. For example, if the viral transcription recognition signal sequence is the COVID-19 viral transcription recognition signal sequence the nucleic acid molecules provided herein would only be expressed in cells infected COVID-19. COVID-19 is merely used as non-limiting example and should not be used to limit the embodiments provided herein.

In some embodiments, the nucleic acid molecules does not comprise sequences (coding or noncoding) for viral polymerase, reverse transcriptase, capsid, envelope, a packaging signal, or a translocation motif. In some embodiments, the recombinant nucleic acid molecule does not comprise sequences (coding or noncoding) for a viral polymerase. In some embodiments, the recombinant nucleic acid molecule does not comprise sequences (coding or noncoding) for a reverse transcriptase. In some embodiments, the recombinant nucleic acid molecule does not comprise sequences (coding or noncoding) for a capsid protein. In some embodiments, the recombinant nucleic acid molecule does not comprise sequences (coding or noncoding) for an envelope protein. In some embodiments, the recombinant nucleic acid molecule does not comprise sequences (coding or noncoding) for a packaging signal. In some embodiments, the recombinant nucleic acid molecule does not comprise sequences (coding or noncoding) for a translocation motif.

The recombinant nucleic acid molecules provided herein can be provided in a vector. The vector, for example, can be a delivery vector or vehicle for delivery to, for example, a mammalian cell, such as a human cell. In some embodiments, the cell is a cyno (e.g. monkey) cell. In some embodiments, the vector is a vector that can deliver the recombinant nucleic acid molecule to a human and a cyno cell. In some embodiments, the vector is a vector that can deliver the recombinant nucleic acid molecule to a human cell, but not a cyno cell. In some embodiments, the vector is a vector that can deliver the recombinant nucleic acid molecule to a cyno cell, but not a human cell.

In some embodiments, the delivery vector or vehicle is a VLP, an adeno-associated virus (AAV), a liposome, a nanoparticle, a micelle, a polymeric vesicle, or a polymersome. In some embodiments, the delivery vector or vehicle is an AAV vector. In some embodiments, the delivery vector or vehicle is a liposome. In some embodiments, the delivery vector or vehicle is a nanoparticle. In some embodiments, the delivery vector or vehicle is a micelle. In some embodiments, the delivery vector or vehicle is a polymeric vesicle. In some embodiments, the delivery vector or vehicle is a polymersome. Non-limiting examples of delivery vectors or vehicles include those that are described in U.S. Patent Application Publication No. 20200206362, U.S. Patent Application Publication No. 20200246267, U.S. Patent Application Publication No. 20170273907, and U.S. Pat. No. 10,556,018, each of which is hereby incorporated by reference in its entirety.

17. Also provided herein are replication incompetent virus-like particle (VLP) that can comprise the recombinant nucleic acid molecules provided herein. In some embodiments, the VLPs comprise an optional translocation motif (TLM) fused to a capsid protein from a virus of interest. For example, the capsid protein can be a hepatitis B viral capsid or a hepatitis D viral capsid or coronavirus fusion protein.

In some embodiments, the recombinant nucleic acid molecule provided by the VLP is a negative strand nucleic acid molecule or a pgRNA nucleic acid molecule encoding a protein of interest. As provided herein, the protein of interest is a chemokine, a cytokine, an apoptosis inducing protein, diphtheria toxin A (or fragment thereof) or a combination thereof. As provided herein, the sequence encoding the protein of interest can be flanked by a first and second viral transcription recognition signal, and further comprising a first promoter upstream (5′) of the first viral transcription recognition signal and a second promoter adjacent and 5′ to the negative strand nucleic acid molecule or pgRNA nucleic acid molecule encoding the protein of interest (e.g. a chemokine, a cytokine, a apoptosis inducing protein, or a combination thereof.) Non-limiting examples of a chemokine, a cytokine, a apoptosis inducing protein, or a combination thereof are provided herein.

In some embodiments, a replication incompetent virus-like particle (VLP) is provided that comprises a recombinant nucleic acid molecule, such as those provided herein (e.g. negative strand nucleic acid molecule or a pgRNA nucleic acid molecule), encoding a protein of interest (e.g., chemokine, a cytokine, a apoptosis inducing protein, or a combination thereof), flanked by a first and second target viral transcription recognition signal, and further comprising a first promoter upstream (5′) of the first viral transcription recognition signal and a second promoter adjacent and 5′ to the negative strand nucleic acid molecule or pgRNA nucleic acid molecule encoding a chemokine, a cytokine, a apoptosis inducing protein, or a combination thereof, wherein the VLP exhibits tropism for the target virally infected cells. The tropism can be controlled by the expression of a viral protein that is expressed on the surface of the VLP as well as controlling the expression of the nucleic acid molecule by the source of the viral transcription recognition signal sequences encoded by the recombinant nucleic acid molecule.

As provided herein, in some embodiments, the negative strand nucleic acid molecule is negative sense RNA, negative sense DNA, single or double strand DNA that expresses a non-coding, negative sense RNA, or pgRNA, or any combination thereof.

In some embodiments, the replication incompetent virus-like particle further comprises a poly A tail sequence downstream (3′) of the negative strand nucleic acid molecule or pgRNA nucleic acid molecule encoding a chemokine, a cytokine, a apoptosis inducing protein, or combination thereof.

In some embodiments, the replication incompetent virus-like particle comprises promoter (e.g. a first promoter and a second promoter). Non-limiting examples of such promoters ae provided herein.

The overall design and the recombinant nucleic acid constructs and replication incompetent virus-like particles, as described herein can be adapted to encompass viruses from Baltimore Groups IV, V, VI, and VII. To target one of the Group IV-VII viruses, a specific viral polymerase recognition signal is utilized that would replace for example, the epsilon recognition signal sequence (SEQ ID NO:1), as described for the HBV example. The Baltimore groups are described as follows:

Baltimore Group IV viruses: possess positive-sense single stranded RNA genomes, including the picornaviruses (which is a family of viruses that includes well-known viruses like Hepatitis A virus, enteroviruses, rhinoviruses, poliovirus, and foot-and-mouth virus), SARS virus, hepatitis C virus, yellow fever virus, and rubella virus. This group also includes the coronaviruses, herpesviruses (Hepatitis E), as well as the flaviviruses, such as Dengue virus, hepatitis C virus, yellow fever virus, and Zika virus.

Baltimore Group V viruses: single stranded RNA viruses; negative sense (e.g. Orthomyxoviruses, Rhabdoviruses).

Baltimore Group VI viruses: positive-sense single stranded RNA viruses that replicate through a DNA intermediate. (e.g. Retroviruses).

Baltimore Group VII viruses: double-stranded DNA viruses that replicate through a single-stranded RNA intermediate.

### NSV Life Cycle and Replication

Negative-strand RNA viruses (NSV, or Baltimore Group V viruses) can be classified into 21 distinct families. The families consisting of nonsegmented genomes include Rhabdo-, Paramyxo-, Filo- and Borna-. Orthomyxo-, Bunya-, Arenaviridae-contain genomes of six to eight, three, or two negative-sense RNA segments, respectively. (See, Palese, P., et al., Proc. Natl. Acad. Sci. USA 93:11354-11358, 1996; and Boritz, Eli et al. Journal of Virology. 73 (8): 6937-6945, 1999).

Many highly prevalent human pathogens such as the respiratory syncytial virus (RSV), parainfluenza viruses, influenza viruses, Ebola virus, Marburg virus are included within the NSV. The life cycle of NSV has a number of steps. The virus first infects the host cell by binding to the host cell receptor through a viral surface glycoprotein. The fusion of the glycoprotein viral membrane with the plasma membrane of the host cell in an acidic environment allows for the release of viral ribonucleoprotein (RNP) complexes into the cytoplasm. Most NSV replicate in the cytoplasm of infected cells. Newly synthesized RNP complexes are assembled with viral structural proteins at the plasma membrane or at membranes of the Golgi apparatus. This is all followed by the release of the newly synthesized viruses.

Regarding the replication and transcription of non-segmented NSV, the genes of these NSV are made up of three regulatory regions: a gene end signal, an intergenic region, and a gene start signal. One example of gene end signals are in a specific virus called the vesicular stomatitis virus (VSV) contains gene end signals that are highly conservative. The intergenic region is highly variable and consists of conserved dinucleotide, trinucleotide, or regions of up to 143 nucleotides. The various lengths of the intergenic regions correlate with transcriptional attenuation, however diverse intergenic regions do not alter the gene expression. The gene start signals are highly specific as the first three nucleotides are critical for the gene expression.

Hepatitis B virus (HBV), a member of the Hepadnaviridae family, is a small DNA virus with unusual features similar to retroviruses. HBV replicates through an RNA intermediate and can integrate into the host genome. The unique features of the HBV replication cycle confer a distinct ability of the virus to persist in infected cells. Virological and serological assays have been developed for diagnosis of various forms of HBV-associated disease and for treatment of chronic hepatitis B infection. HBV infection leads to a wide spectrum of liver disease ranging from acute (including fulminant hepatic failure) to chronic hepatitis, cirrhosis, and hepatocellular carcinoma. Acute HBV infection can be either asymptomatic or present with symptomatic acute hepatitis. Most adults infected with the virus recover, but 5%-10% are unable to clear the virus and become chronically infected. Many chronically infected persons have mild liver disease with little or no long-term morbidity or mortality. Other individuals with chronic HBV infection develop active disease, which can progress to cirrhosis and liver cancer. Additionally, some individuals are infected with other hepatitis viruses in addition to HBV, such as hepatitis A, (HAV), hepatitis C (HCV), hepatitis D (HDV), or hepatitis E (HEV). Thus, treating HBV will assist in overcoming these co-infections, especially HDV, which requires HBV for its replication. Treating HBV will also lessen the likelihood of oncogenic abnormalities and cirrhosis.

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein and unless otherwise indicated, the term “about” is intended to mean±5% of the value it modifies. Thus, about 100 means 95 to 105. Additionally, the term “about” modifies a term in a series of terms, such as “about 1, 2, 3, 4, or 5” it should be understood that the term “about” modifies each of the members of the list, such that “about 1, 2, 3, 4, or 5” can be understood to mean “about 1, about 2, about 3, about 4, or about 5.” The same is true for a list that is modified by the term “at least” or other quantifying modifier, such as, but not limited to, “less than,” “greater than,” and the like.

As used herein and in the appended claims, the singular forms “a”, “an” and “the” include plural reference unless the context clearly dictates otherwise.

As used herein, the terms “comprise,” “have,” “has,” and “include” and their conjugates, as used herein, mean “including but not limited to.” While various compositions, and methods are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

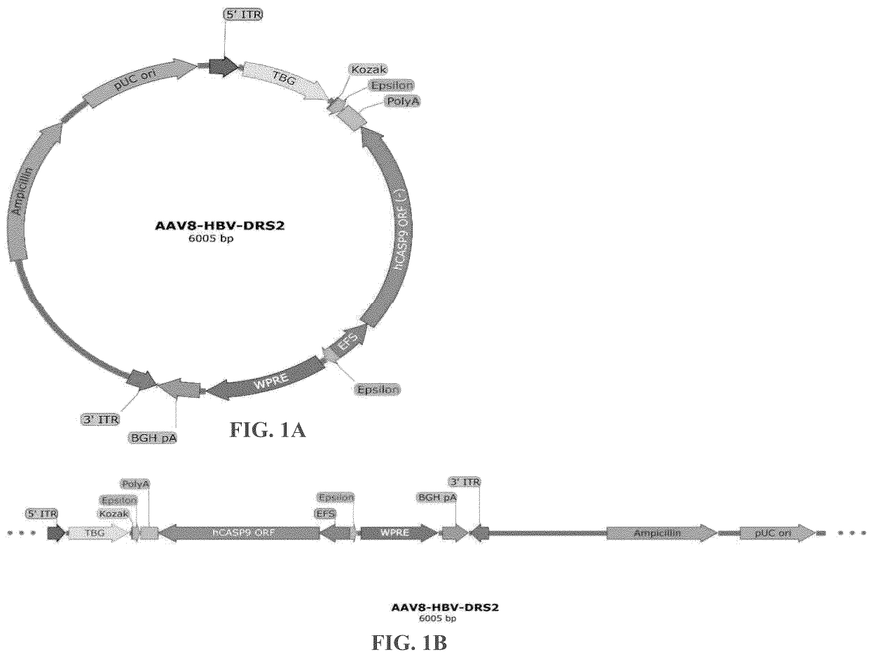

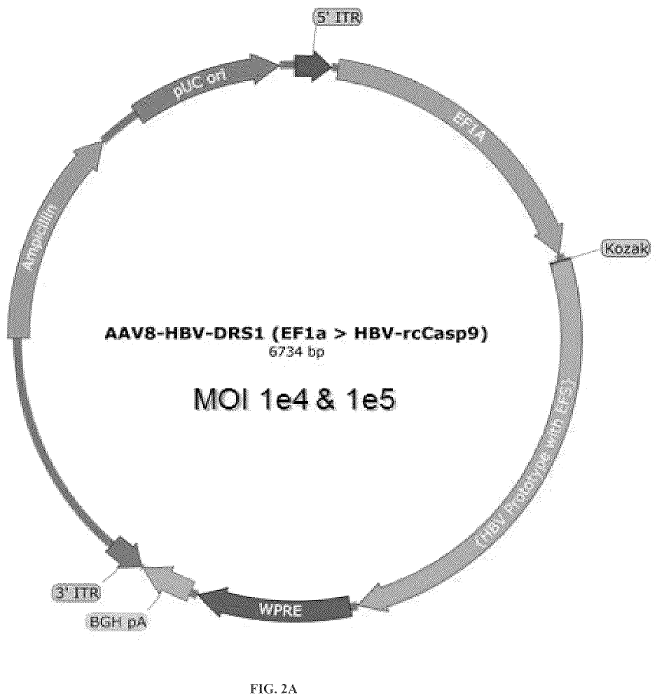

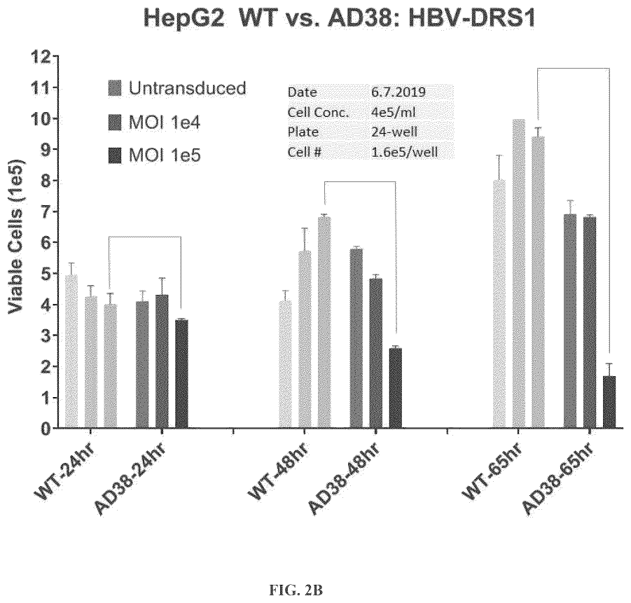

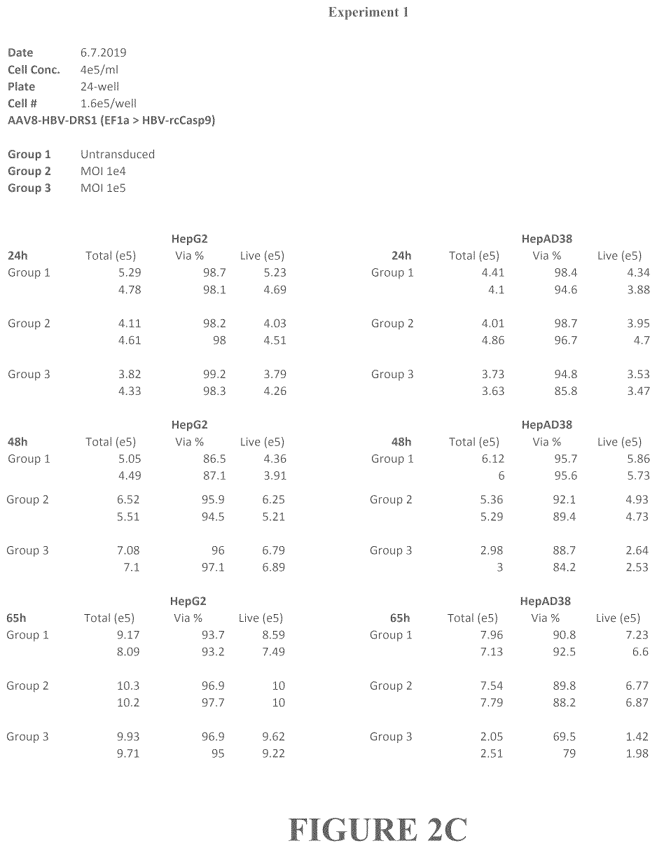

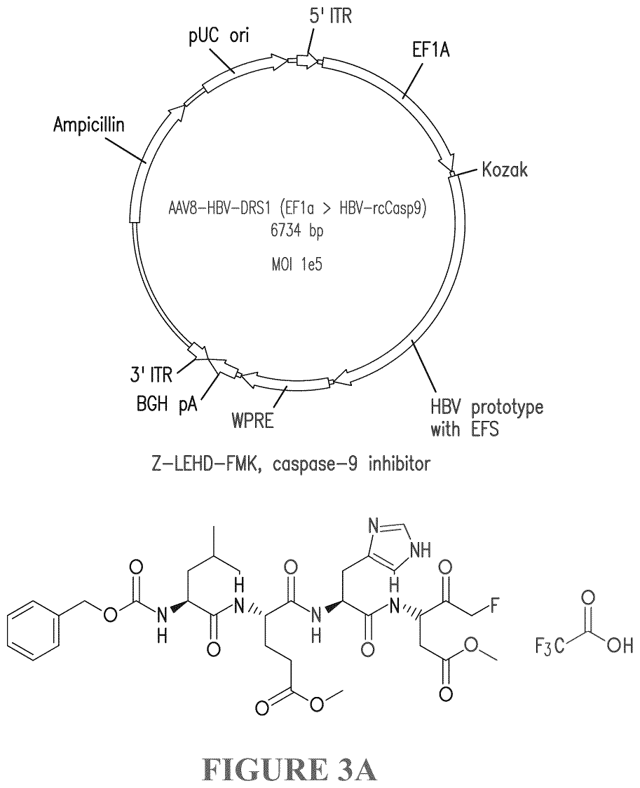

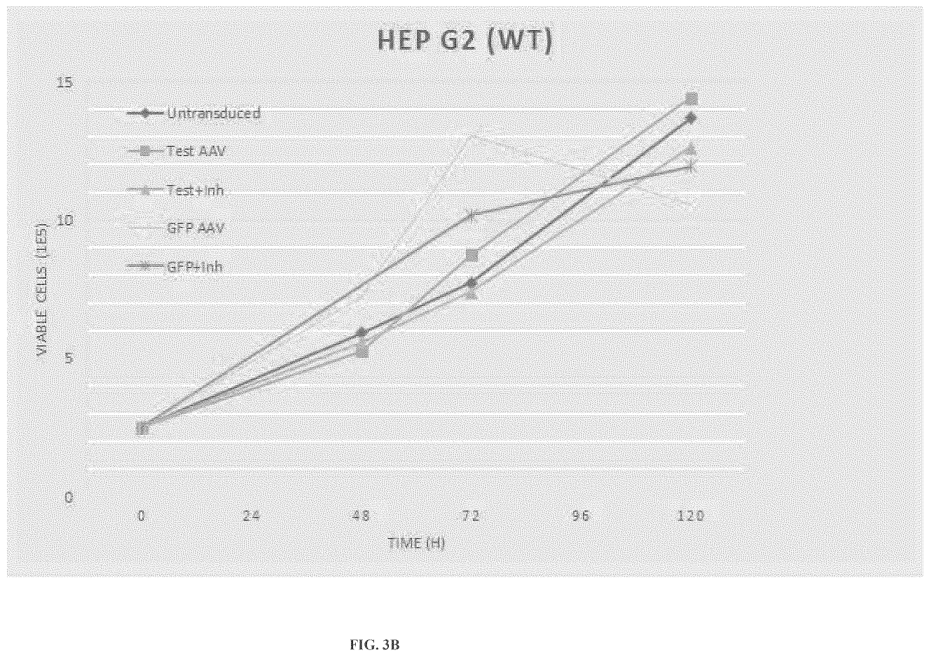

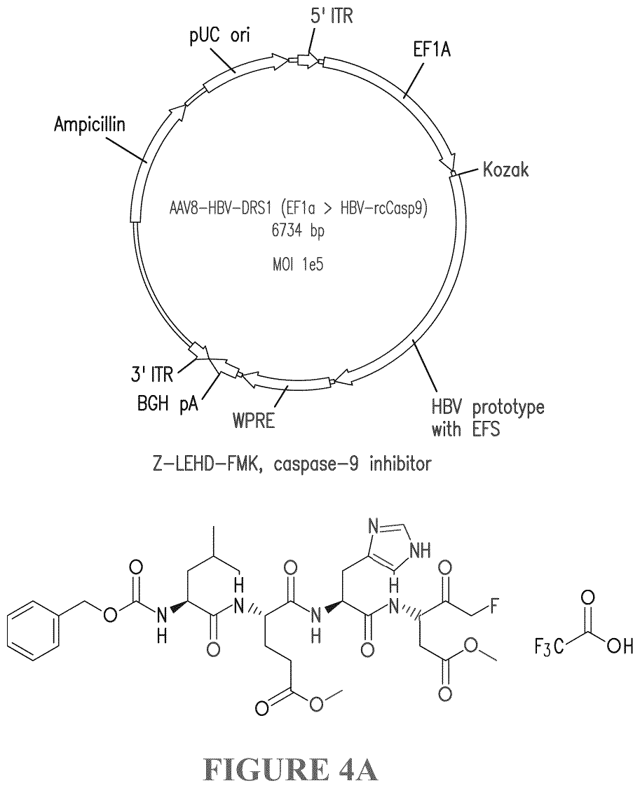

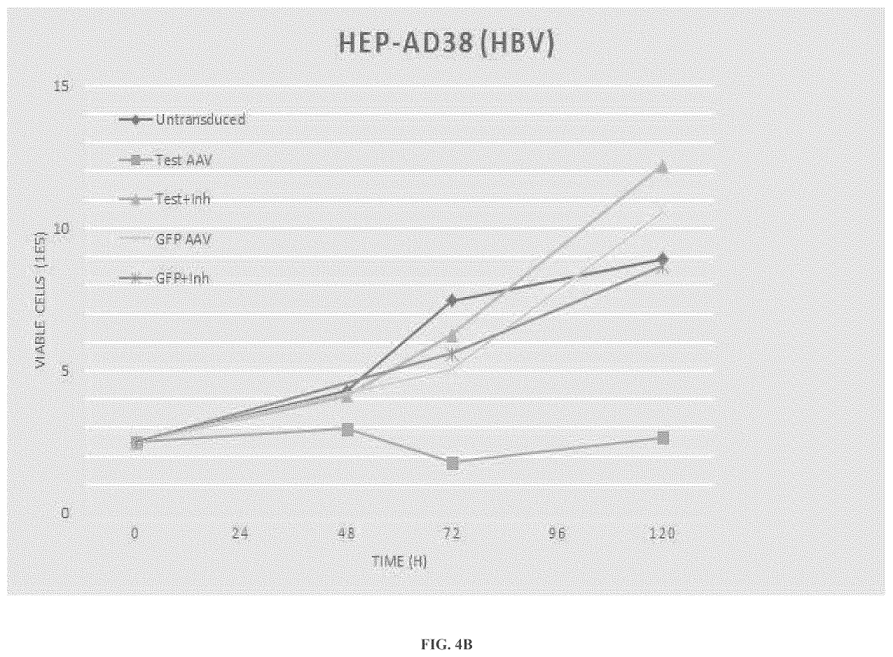

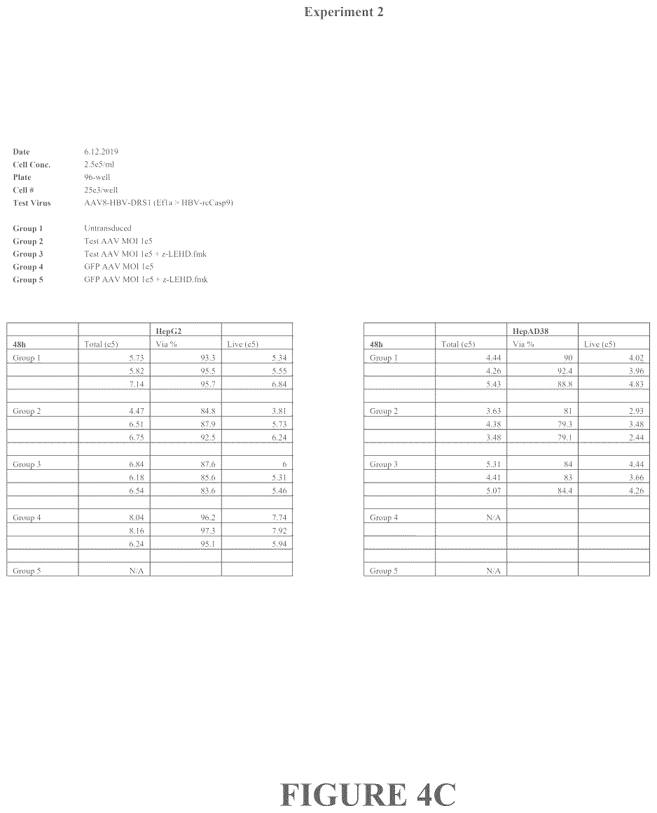

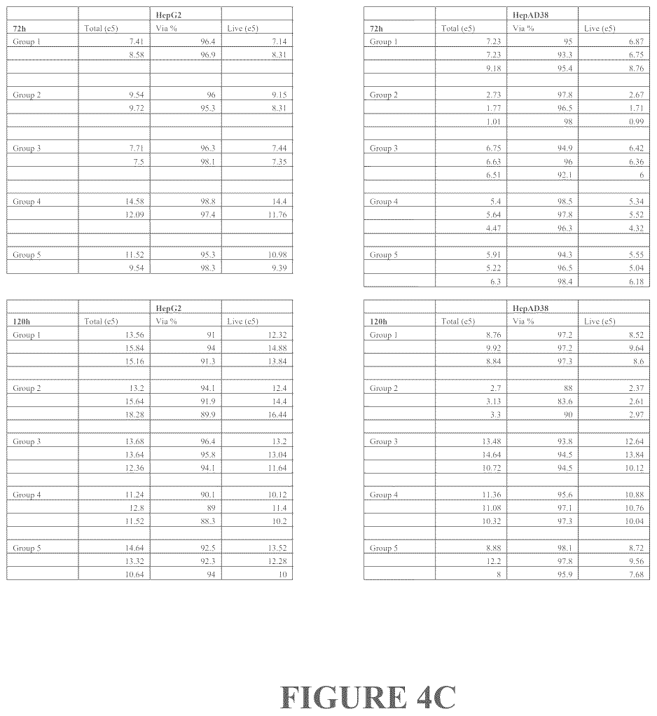

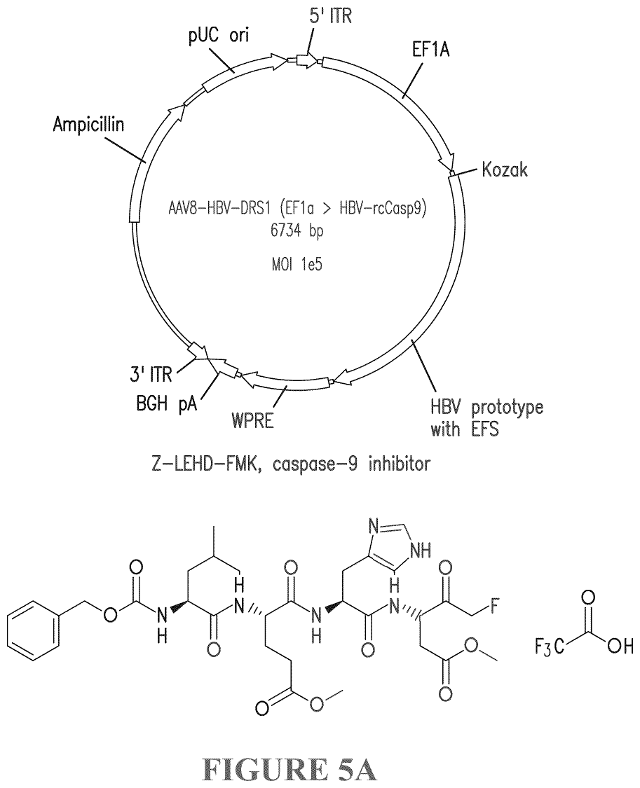

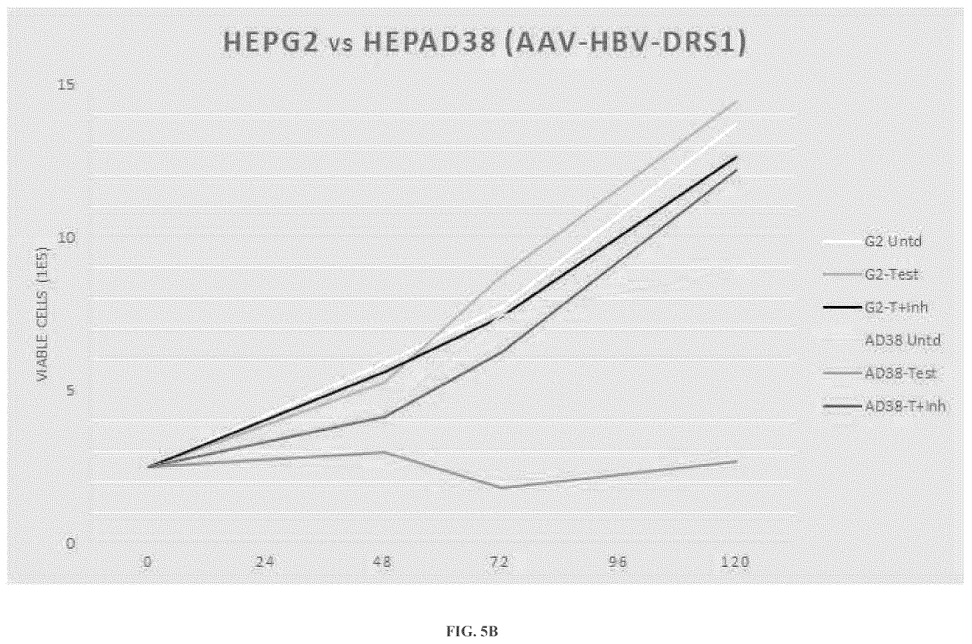

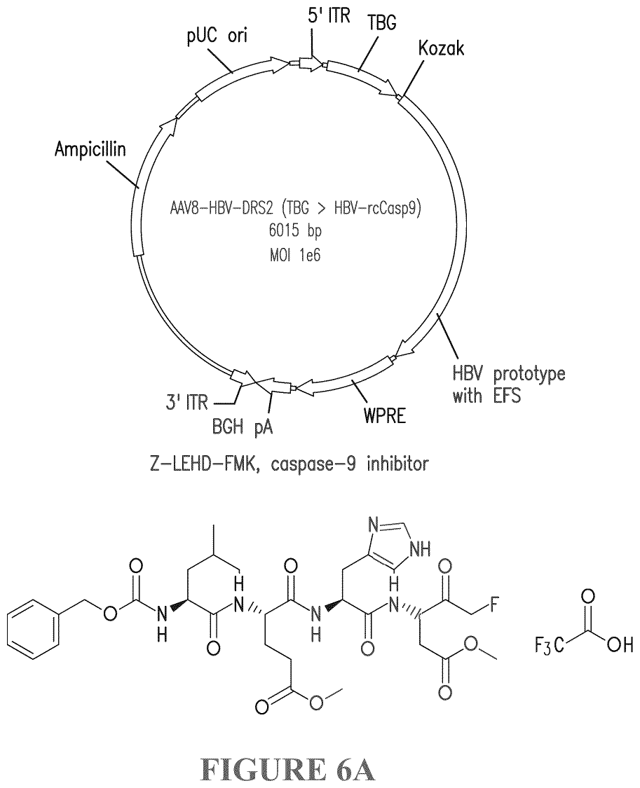

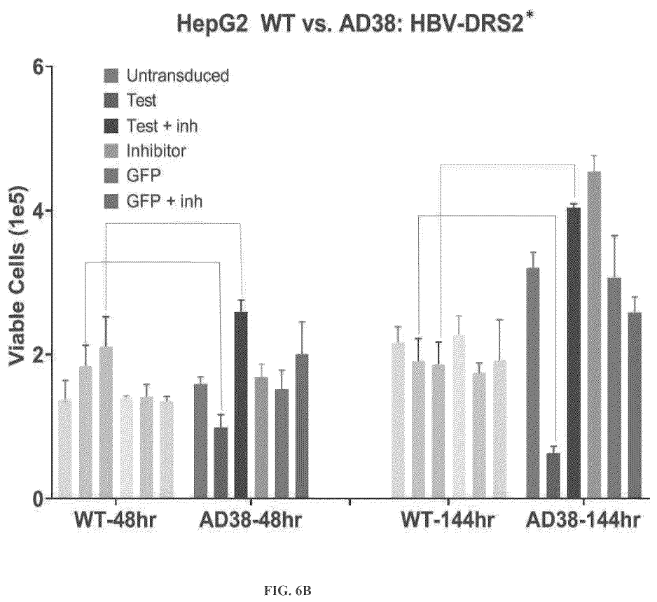

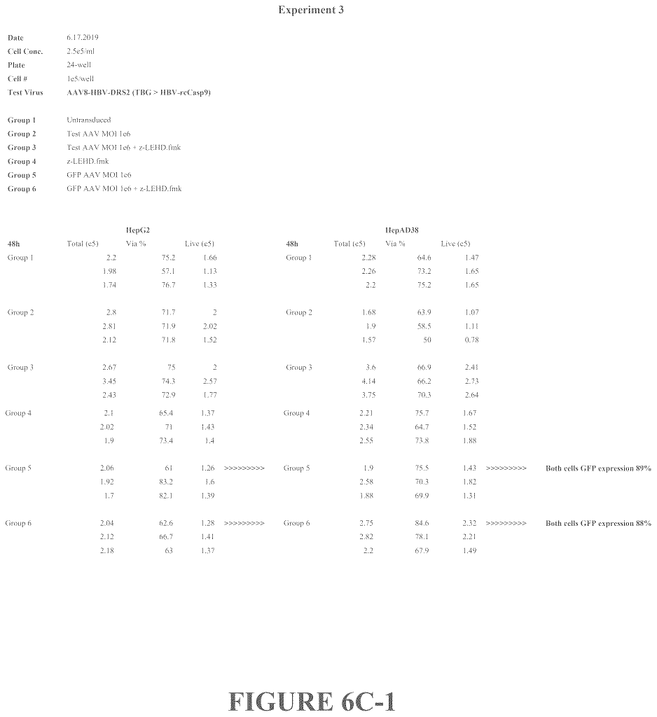

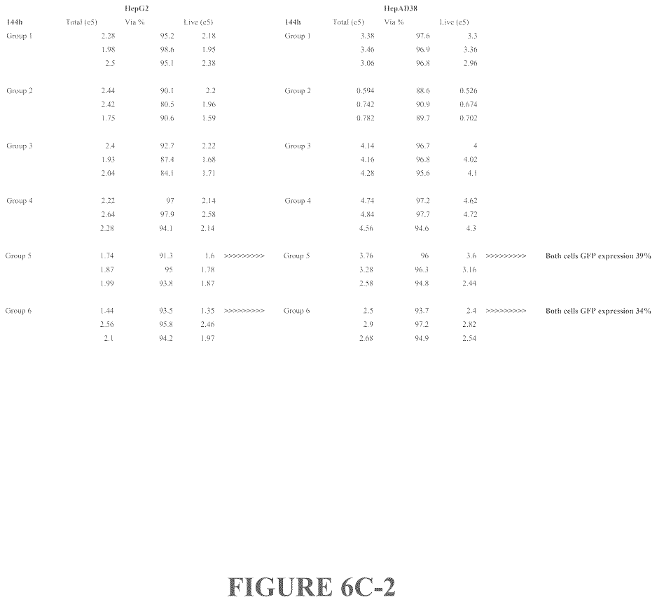

Vector maps of the exemplary constructs used to test recombinant variants described herein are shown in FIG. 1A, FIG. 1B, FIG. 2A, FIG. 3A, FIG. 4A, FIG. 5A, FIG. 6A, FIG. 7, AND FIG. 8. While graphs of results testing these constructs are shown in FIG. 2B, FIG. 3B, FIG. 4B FIG. 4C, FIG. 5B, FIG. 6B and FIG. 6C. Exemplary sequences useful in these constructs are provided in SEQ ID NOs:1-9. These are non-limiting examples and can be modified or tailored based on the virus of interest to be treated.

The terms “co-administration” or the like, are meant to encompass administration of the selected therapeutic agents to a single patient, and are intended to include treatment regimens in which the agents are administered by the same or different route of administration or at the same or different time.

As used herein, the term “agonist” refers to a compound, the presence of which results in a biological activity of a protein that is the same as the biological activity resulting from the presence of a naturally occurring ligand for the protein.

As used herein, the term “partial agonist” refers to a compound the presence of which results in a biological activity of a protein that is of the same type as that resulting from the presence of a naturally occurring ligand for the protein, but of a lower magnitude.

As used herein, the term “antagonist” refers to a compound, the presence of which results in a decrease in the magnitude of a biological activity of a protein. In certain embodiments, the presence of an antagonist results in complete inhibition of a biological activity of a protein. In certain embodiments, an antagonist is an inhibitor.

“Administering” when used in conjunction with a therapeutic composition (e.g. recombinant nucleic acid constructs and replication incompetent virus-like particles and compositions comprising these products) means to administer a therapeutic directly into or onto a target tissue or to administer a therapeutic to a patient whereby the therapeutic positively impacts the tissue to which it is targeted.

The term “subject” or “patient” as used herein includes, but is not limited to, humans and non-human vertebrates such as wild, domestic, and farm animals. In certain embodiments, the subject or patient described herein is an animal. In certain embodiments, the subject or patient is a mammal. In certain embodiments, the subject is a human. In certain embodiments, the subject or patient is a non-human animal. In certain embodiments, the subject or patient is a non-human mammal. In certain embodiments, the subject or patient is a domesticated animal, such as a dog, cat, cow, pig, horse, sheep, or goat. In certain embodiments, the subject or patient is a companion animal such as a dog or cat. In certain embodiments, the subject or patient is a livestock animal such as a cow, pig, horse, sheep, or goat. In certain embodiments, the subject or patient is a zoo animal. In another embodiment, the subject or patient is a research animal such as a rodent, dog, or non-human primate. In certain embodiments, the subject or patient is a non-human transgenic animal such as a transgenic mouse or transgenic pig.

The term “inhibit” includes the administration of a therapeutic of embodiments herein to prevent the onset of the symptoms, alleviating the symptoms, or eliminating the disease, condition or disorder.

By “pharmaceutically acceptable”, it is meant the carrier, diluent or excipient must be compatible with the other ingredients of the therapeutic and not deleterious to the recipient thereof.

The terms “treat,” “treated,” or “treating” as used herein refers to both therapeutic treatment and prophylactic or preventative measures, wherein the object is to inhibit, prevent or slow down (lessen) an undesired physiological condition, disorder or disease, or to improve, inhibit, or otherwise obtain beneficial or desired clinical results. For the purposes of this invention, beneficial or desired clinical results include, but are not limited to, improvement or alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. The antibodies may exist in a variety of forms including, for example, polyclonal antibodies, monoclonal antibodies, Fv, Fab and F(ab)2, as well as single chain antibodies and humanized antibodies.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the embodiments include, but are not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tissue sample suspected of containing a virus, a cell or a biological fluid.

The term “antigen” as used herein is defined as a molecule that provokes an immune response. This immune response may involve either antibody production, or the activation of specific immunologically-competent cells, or both.

As used herein, the term “ex vivo” refers to “outside” the body.

A “disease” is a state of health of a subject wherein the subject cannot maintain homeostasis, and wherein if the disease is not ameliorated then the animal's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

In some embodiments, protein is at least, or about, 90, 91, 92, 93, 94, 95, 96, 97, 98, 99% homologous to the sequences provided herein. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated” biological component (such as a nucleic acid, protein or cell) has been substantially separated or purified away from other biological components (such as cell debris, other proteins, nucleic acids or cell types). Biological components that have been “isolated” include those components purified by standard purification methods.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

As used herein, recombinant generally refers to the following: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

As used herein, the following abbreviations for the commonly occurring nucleic acid bases are used. “A” refers to adenosine, “C” refers to cytosine, “G” refers to guanosine, “T” refers to thymidine, and “U” refers to uridine.

The term “leukocytes” or “white blood cell” as used herein refers to any immune cell, including monocytes, neutrophils, eosinophils, basophils, and lymphocytes. The term “lymphocytes” as used herein refer to cells commonly found in lymph, and include natural killer cells (NK cells), T-cells, and B-cells. It will be appreciated by one of skill in the art that the above listed immune cell types can be divided into further subsets.

The term “tumor infiltrating leukocytes” as used herein refers to leukocytes that are present in a solid tumor.

The term “blood sample” as used herein refers to any sample prepared from blood, such as plasma, blood cells isolated from blood, and so forth.

The term “purified sample” as used herein refers to any sample in which one or more cell subsets are enriched. A sample may be purified by the removal or isolation of cells based on characteristics such as size, protein expression, and so forth.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compositions, and additional pharmaceutical agents.

In general, the nature of a suitable carrier or vehicle for delivery will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In some embodiments, compositions, whether they be solutions, suspensions or other like form, may include one or more of the following: DMSO, sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Diseases that the compositions and methods described herein can treat include microbial infections such as a viral infection.

By a “viral infection” is meant an infection caused by the presence of a virus in the body. Viral infections include chronic or persistent viral infections, which are viral infections that are able to infect a host and reproduce within the cells of a host over a prolonged period of time-usually weeks, months or years, before proving fatal.

Viruses giving rise to chronic infections include, for example, the human papilloma viruses (HPV), Herpes simplex, and other herpes viruses, the viruses of hepatitis B and C as well as other hepatitis viruses, human immunodeficiency virus, and the measles virus, all of which can produce important clinical diseases. Prolonged infection may ultimately lead to the induction of disease which may be, e.g., in the case of hepatitis C virus liver cancer, fatal to the patient. Other chronic viral infections which may be treated in accordance with the present invention include any Group V-VII virus that utilizes a virus-specific polymerase that can be used as an activating enzyme for transcribing the inactive recombinant nucleotide vector/VLP as described herein.

In certain embodiments, the recombinant nucleic acid constructs and replication incompetent virus-like particles, or compositions comprising such constructs/particles can be administered simultaneously with anti-microbial, anti-viral and/or other therapeutic agents. Alternatively, constructs/particles or composition comprising such constructs/particles can be administered at selected times in advance of times when anti-microbial, anti-viral and other therapeutic agents are administered.

Antivirals include, but are not limited to, ritonavir, acyclovir, cidofovir, ganciclovir, foscarnet, zidovudine, ribavirin, and hydroxychloroquine.

Antivirals further include, and are not limited to HIV treatments such as:
small molecule HIV fusion or entry inhibitors include: bevirimat (DSB;PA-457); Vicriviroc, Maraviroc (a chemokine receptor antagonist” or a “CCR5 inhibitor”), T-20 (enfuvirtide, Fuzeon, developed by Roche and Trimeris), TRI-1144, and TRI-999 (See, Qian, K et al, Med Res Rev. 2009 March; 29(2):369-393, and Haggani and Tilton, Antiviral Res. 2013 May; 98(2):158-70). Similarly, examples of anti-HIV mAbs include those against CCR5 and a CD4, and specifically: Ibalizumab (trade name Trogarzo) is a non-immunosuppressive humanized monoclonal antibody that binds CD4; PRO 140 is a humanized monoclonal antibody targeted against the CCR5.

Antiviral agents for combination treatment can include any one or combination of: an HBV polymerase inhibitor, interferon, TLR modulators such as TLR-7 agonists or TLR-9 agonists, therapeutic vaccines, immune activator of certain cellular viral RNA sensors, viral entry inhibitor, viral maturation inhibitor, distinct capsid assembly modulator, antiviral compounds of distinct or unknown mechanism.

Antiviral agents can also be any one or combination of: 3TC, FTC, L-FMAU, interferon, adefovir dipivoxil, entecavir, telbivudine (L-dT), valtorcitabine (3′-valinyl L-dC), .beta.-D-dioxolanyl-guanine (DXG), .beta.-D-dioxolanyl-2,6-diaminopurine (DAPD), .beta.-D-dioxolanyl-6-chloropurine (ACP), famciclovir, penciclovir, lobucavir, ganciclovir, ribavirin, tenofovir, bictegravir, emtricitabine, Biktarvy, and any combination thereof.

In some embodiments, a “therapeutically effective amount” is an amount of recombinant nucleic acid constructs and replication incompetent virus-like particles, or composition comprising such constructs/particles, as described herein that results in a reduction in viral titer by at least 2.5%, at least 5%, at least 10%, at least 15%, at least 25%, at least 35%, at least 45%, at least 50%, at least 75%, at least 85%, by at least 90%, at least 95%, or at least 99% in a subject/patient/animal administered the recombinant nucleic acid constructs and replication incompetent virus-like particles, or composition comprising such constructs/particles and treated with a related method described herein, relative to the viral titer or microbial titer in an animal or group of animals (e.g., two, three, five, ten or more animals) not administered a recombinant nucleic acid constructs and replication incompetent virus-like particles, or composition comprising such constructs/particles of the invention.

### Examples of Methods of Viral Vector-Mediated Transfer

In certain embodiments, a recombinant nucleic acid construct is incorporated into a viral like particle (defective in its ability to self-replicate) to mediate gene transfer to a cell. Typically, the virus simply will be exposed to the appropriate host cell under physiologic conditions, permitting uptake of the virus. (See, U.S. Pat. No. 9,089,520) The present methods can be adapted to utilize a variety of viral vectors or viral-like particles to deliver the recombinant constructs to a desired cellular target, as discussed below, and includes adenoviral vector systems, which are optimized to be incompetent, or non-replicating VLPs.

### 1. Adenovirus

Adenovirus is particularly suitable for use as a gene transfer vector because of its mid-sized DNA genome, ease of manipulation, high titer, wide target-cell range, and high infectivity. The roughly 36 kb viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, M. J. (1990) Radiother Oncol., 19, 197-218). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence, which makes them useful for translation.

In certain instances, it is helpful to maximize the carrying capacity of AAVs so that large segments of DNA can be included. It also is very desirable to reduce the toxicity and immunologic reaction associated with certain adenoviral products. The two goals are, to an extent, coterminous in that elimination of adenoviral genes serves both ends.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay, R. T., et al., J Mol. Biol. 1984 Jun. 5; 175(4):493-510). Therefore, deletion of these elements in an adenoviral vector will prevent independent replication.

In addition, the packaging signal for viral encapsulation is localized between 194-385 bp (0.5-1.1 map units) at the left end of the viral genome (Hearing et al., J. (1987) Virol., 67, 2555-2558). This signal mimics the protein recognition site in bacteriophage lambda DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., Gene, 101:195-202, 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

To produce VLP/replication-deficient adenoviral vectors in vitro, these deficient vectors can be complemented, in trans, by helper virus. However, this does not permit isolation of the replication-deficient vectors, however, since the presence of helper virus, needed to provide replicative functions, would contaminate any preparation. Thus, an additional element that would add specificity to the replication and/or packaging of the replication-deficient vector is needed. That element derives from the packaging function of adenovirus.

It has been shown that a packaging signal for adenovirus exists in the left end of the conventional adenovirus map (Tibbetts et. al. (1977) Cell, 12, 243-249). Later studies showed that a mutant with a deletion in the E1A (194-358 bp) region of the genome grew poorly even in a cell line that complemented the early (E1A) function (Hearing and Shenk, (1983) J. Mol. Biol. 167, 809-822). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved toward the interior of the Ad5 DNA molecule (Hearing et al., J. (1987) Virol., 67, 2555-2558).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals is packaged selectively when compared to the helpers. If the preference is great enough, stocks approaching homogeneity may be achieved.

To improve the tropism of ADV constructs for particular tissues or species, the receptor-binding fiber sequences can often be substituted between adenoviral isolates. For example the Coxsackie-adenovirus receptor (CAR) ligand found in adenovirus 5 can be substituted for the CD46-binding fiber sequence from adenovirus 35, making a virus with greatly improved binding affinity for human hematopoietic cells. The resulting “pseudotyped” virus, Ad5f35, has been the basis for several clinically developed viral isolates. Moreover, various biochemical methods exist to modify the fiber to allow re-targeting of the virus to target cells. Methods include use of bifunctional antibodies (with one end binding the CAR ligand and one end binding the target sequence), and metabolic biotinylation of the fiber to permit association with customized avidin-based chimeric ligands. Alternatively, one could attach ligands (e.g. anti-CD205 by heterobifunctional linkers (e.g. PEG-containing), to the adenovirus particle.

### 2. Retrovirus

The retroviruses are a group of single-stranded RNA viruses characterized by an ability to convert their RNA to double-stranded DNA in infected cells by a process of reverse-transcription (Coffin, (1990) In: Virology, ed., New York: Raven Press, pp. 1437-1500). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and psi components is constructed (Mann et al., (1983) Cell, 33, 153-159). When a recombinant plasmid containing a human cDNA, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas, J. F., and Rubenstein, J. L. R., (1988) In: Vectors: a Survey of Molecular Cloning Vectors and Their Uses, Rodriquez and Denhardt, Eds.). Nicolas and Rubenstein; Temin et al., (1986) In: Gene Transfer, Kucherlapati (ed.), New York: Plenum Press, pp. 149-188; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., (1975) Virology, 67, 242-248). An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, may this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., (1989) Proc. Nat'l Acad. Sci. USA, 86, 9079-9083). Using antibodies against major histocompatibility complex class I and class II antigens, the infection of a variety of human cells that bore those surface antigens was demonstrated with an ecotropic virus in vitro (Roux et al., 1989).

### 3. Adeno-Associated Virus

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low-level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., J. Virol., 61:3096-3101 (1987)), or by other methods, including but not limited to chemical or enzymatic synthesis of the terminal repeats based upon the published sequence of AAV. It can be determined, for example, by deletion analysis, the minimum sequence or part of the AAV ITRs which is required to allow function, i.e., stable and site-specific integration. It can also be determined which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, (1995) Ann. N.Y. Acad. Sci., 770; 79-90; Chatteijee, et al., (1995) Ann. N.Y. Acad. Sci., 770, 79-90; Ferrari et al., (1996) J. Virol., 70, 3227-3234; Fisher et al., (1996) J. Virol., 70, 520-532; Flotte et al., Proc. Nat'l Acad. Sci. USA, 90, 10613-10617, (1993); Goodman et al. (1994), Blood, 84, 1492-1500; Kaplitt et al., (1994) Nat'l Genet., 8, 148-153; Kaplitt, M. G., et al., Ann Thorac Surg. 1996 December; 62(6):1669-76; Kessler et al., (1996) Proc. Nat'l Acad. Sci. USA, 93, 14082-14087; Koeberl et al., (1997) Proc. Nat'l Acad. Sci. USA, 94, 1426-1431; Mizukami et al., (1996) Virology, 217, 124-130).

AAV-mediated efficient gene transfer and expression in the lung has led to clinical trials for the treatment of cystic fibrosis (Carter and Flotte, 1995; Flotte et al., Proc. Nat'l Acad. Sci. USA, 90, 10613-10617, (1993)). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., (1996) J. Virol., 70, 520-532; Flotte et al., 1993; Kaplitt et al., 1994; 1996; Koeberl et al., 1997; McCown et al., (1996) Brain Res., 713, 99-107; Ping et al., (1996) Microcirculation, 3, 225-228; Xiao et al., (1996) J. Virol., 70, 8098-8108).

The challenges associated with liver-directed gene therapy are efficient targeting of hepatocytes, stability of the vector genome, and persistent high level expression. Many of these obstacles can be overcome with adeno-associated viral (AAV) gene transfer vectors. The first AAV gene transfer vector developed for in vivo use was based on the AAV2 serotype. AAV2 has a broad tropism and transduces many cell types, including hepatocytes, relatively efficiently in vivo. The capsid protein confers the serological profile and at least 12 primate AAV serotypes have already been characterized. Importantly, pseudotyping a recombinant AAV vector with different capsid proteins can dramatically alter the tropism. Both AAV8 and AAV9 have higher affinities for hepatocytes when compared to AAV2. In particular, AAV8 can transduce 3-4 fold more hepatocytes and deliver 3-4 fold more genomes per transduced cell when compared to AAV2 (See, Mark S. Sands, Methods Mol. Biol. 2011; 807: 141-157). Depending on the dose, AAV8 can transduce up to 90-95% of hepatocytes in the mouse liver following intraportal vein injection. Interestingly, comparable levels of transduction can be achieved following intravenous injection. Direct intraparenchymal injection of an AAV vector also mediates relatively high level long term expression. Additional specificity can be conferred by using liver-specific promoters in conjunction with AAV8 capsid proteins. In addition to treating primary hepatocyte defects, immune reactions to transgene products can be minimized by circumventing the fixed tissue macrophages of the liver, Kupffer cells, and limiting expression to hepatocytes. The ability to target hepatocytes by virtue of the AAV serotype and the use of liver-specific promoters allows testing novel therapeutic approaches.

### 4. Lentiviral Vectors

In certain embodiments, the recombinant nucleic acid or replication incompetent VLPs are transduced into the target cells, by electroporation, or by transfection of nucleic acids, proteins, site-specific nucleases, self-replicating RNA viruses or integration-deficient lentiviral vectors. (for such vectors see, U.S. Pat. No. 10,131,876).

In certain embodiments, transduction is performed with lentiviruses, gamma-, alpha-retroviruses or adenoviruses or with electroporation or transfection by nucleic acids (DNA, mRNA, miRNA, antagomirs, ODNs), proteins, site-specific nucleases (zinc finger nucleases, TALENs, CRISP/R), self-replicating RNA viruses (e.g. equine encephalopathy virus) or integration-deficient lentiviral vectors.

In further embodiments, delivery of the recombinant nucleic acid or replication incompetent VLPs may be performed by transducing said cells with lentiviral vectors (See, Cockrell Adam S et al., “Gene delivery by lentivirus vectors”, Molecular Biotechnology, vol. 36, No. 3, July 2007.)

Lentiviral vectors with the VSVG pseudotype enable efficient transduction under automated manufacturing method. However, the present methods are entirely suitable for the use of any type of lentiviral vector (with e.g. measles virus (ML-LV), gibbon ape leukaemia virus (GALV), feline endogenous retrovirus (RD 114), baboon endogenous retrovirus (BaEV) derived pseudotyped envelopes). Other viral vectors such as gamma or alpha retroviral vectors can be used. Transduction enhancer reagents can be added when necessary using the automated manufacturing described in this invention.

### 5. Other Viral Vectors

Other viral vectors can be employed as expression constructs in the present methods and compositions. Vectors derived from viruses such as vaccinia virus (Ridgeway, (1988) In: Vectors: A survey of molecular cloning vectors and their uses, pp. 467-492; Baichwal and Sugden, (1986) In, Gene Transfer, pp. 117-148; Coupar et al., Gene, 68:1-10, 1988) canary poxvirus, and herpes viruses are employed. These viruses offer several features for use in gene transfer into various mammalian cells.

### Methods for Treating a Disease

The present methods also encompass methods of treatment or prevention of a viral disease or condition where administration of recombinant nucleic acids, VLP products, or pharmaceutical compositions can be delivered in various effective amounts.

The term “unit dose” as it pertains to the inoculum refers to physically discrete units suitable as unitary dosages for mammals, each unit containing a predetermined quantity of pharmaceutical composition calculated to produce the desired immunogenic effect in association with the required diluent. The specifications for the unit dose of an inoculum are dictated by and are dependent upon the unique characteristics of the pharmaceutical composition and the particular immunologic effect to be achieved.

An effective amount of the recombinant nucleic acids, VLP products or pharmaceutical compositions thereof, would be an amount, such that over 60%, 70%, 80%, 85%, 90%, 95%, or 97% of the virally infected, e.g. HBV, Covid-19, etc. infected cells are killed. The term is also synonymous with “sufficient amount.”

The effective amount for any particular application can vary depending on such factors as the disease or condition being treated, the particular composition being administered, the size of the subject, and/or the severity of the disease or condition. One can empirically determine the effective amount of a particular composition presented herein without necessitating undue experimentation.

The terms “contacted” and “exposed,” when applied to a cell, tissue or organism, are used herein to describe the process by which the pharmaceutical composition and/or another agent, such as for example an anti-viral agent, are delivered to a target cell, tissue or organism or are placed in direct juxtaposition with the target cell, tissue or organism. To achieve cell killing or stasis, recombinant nucleic acids, VLP products or pharmaceutical compositions thereof, and/or additional agent(s) are delivered to one or more cells in a combined amount effective to kill the virally infected cell(s) or prevent them from dividing. The administration of the recombinant nucleic acid, VLP products, or pharmaceutical composition may precede, be co-current with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other aspects, one or more agents may be administered substantially simultaneously, about 1 minute, to about 24 hours to about 7 days to about 1 to about 8 weeks or more, and any range derivable therein, prior to and/or after administering the recombinant nucleic acid, VLP, or pharmaceutical products. Yet further, various combination regimens of the pharmaceutical composition presented herein and one or more agents may be employed.

### Formulations and Routes for Administration to Patients

Where clinical applications are contemplated, it will be necessary to prepare pharmaceutical compositions—expression constructs, expression vectors, fused proteins, transfected or transduced cells, in a form appropriate for the intended application. Generally, this will entail preparing compositions that are essentially free of pyrogens, as well as other impurities that could be harmful to humans or animals.

The recombinant nucleic acids, VLP products or pharmaceutical compositions thereof, may be delivered, for example at doses of about 1-5 million particles per dose. Vials or other containers may be provided containing the product, for example, a volume per vial of about 0.25 ml to about 10 ml, for example, about 0.25, 0.5, 1, 1.5, 2, 2.5, 3, 3.5, 4, 4.5, 5, 5.5, 6, 6.5, 7, 7.5, 8, 8.5, 9, 9.5, or 10 ml, for example, about 2 ml.

One may generally desire to employ appropriate salts and buffers when recombinant nucleic acid or VLP products are introduced into a patient. The phrase “pharmaceutically or pharmacologically acceptable” refers to molecular entities and compositions that do not produce adverse, allergic, or other untoward reactions when administered to an animal or a human. A pharmaceutically acceptable carrier includes any and all solvents, dispersion media, coatings, antibacterial and antifungal agents, isotonic and absorption delaying agents and the like. The use of such media and agents for pharmaceutically active substances is known. Except insofar as any conventional media or agent is incompatible with the vectors or cells, its use in therapeutic compositions is contemplated. Supplementary active ingredients also can be incorporated into the compositions.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media can be employed. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations may meet sterility, pyrogenicity, and general safety and purity standards as required by FDA Office of Biologics standards.

The compositions may be formulated for aerosolized delivery to a subject. For aerosol delivery, the compositions described may be formulated in aqueous solutions such as water or in physiologically compatible buffers such as Hanks's solution, Ringer's solution, or physiological saline buffer. The solution may contain one or more formulatory agents such as suspending, stabilizing or dispersing agents.

Delivery systems of the disclosure that deliver the polynucleotides of the disclosure to a desired cell of a subject are not limited to the VLPs of the disclosure. In some embodiments, the delivery vector comprises an adeno-associated virus (AAV) vector, a liposome, a nanoparticle, a micelle, a polymeric vesicle or a polymersome.

In some embodiments, the delivery vector comprises a vector derived from an AAV. AAV vectors are among the most frequently used vectors for gene therapy. AAV is a small, non-enveloped Class II virus, whose genome is encoded in one long single stranded DNA molecule. AAV derived vectors have the ability to attach and enter a target cell, transfer genetic material to the nucleus, and have that information be expressed for sustained periods of time with a general lack of toxicity. AAV derived vectors typically do not encode AAV components necessary for site specific integration into the host genome, and typically persist as extrachromosomal elements. AAV vectors are also capable, to some degree, of selective tissue and organ targeting. Thus, AAV vectors are a possible delivery mechanism for the single stranded DNA polynucleotides of the disclosure to a desired cell of a subject.

In some embodiments, the recombinant polynucleotides of the disclosure can be packaged into a non-viral delivery system to be delivered to a desired target cell of a subject. In some embodiments, the polynucleotides of the disclosure are packaged into a nanoparticle, a micelle, a polymeric vesicle or a polymersome to be delivered to a desired target cell of a subject. In some embodiments, the these products can be formulated into an aerosol for delivery by inhaler, or by nanoparticle. Additional routes of delivery include: topical, transdermal, intravenous, sub-cutaneous, and intrathecal delivery. In some embodiments, the nanoparticle encapsulates the ssRNA encoding the therapeutic, which can then be administered systemically or locally, such as by aerosol delivery. Aerosol delivery can be used to deliver the therapeutic to the lungs of a patient that is infected with a virus as provided herein, such as a coronavirus.

Additionally, in certain patients, it is expected that this treatment would be repeated periodically to reduce or eliminate any remaining virus/virions. Such periodic treatment can vary from once every week, once every 2 weeks, once every 3 weeks, once a month, to once every two months, to once every 3 months, to once every 4 months, to once every 5 months, to once every 6 months, or once every 7 months, or once every 8 months, or once every 9 months, or once every 10 months, or every 11 months, or once annually as a maintenance treatment for as long as the patient requires to achieve stable or undetectable disease.

### Combination and Alternation Therapy

It has been recognized that drug-resistant variants of many viral infections including HIV, HBV and HCV can emerge after prolonged treatment with an antiviral agent. Drug resistance most typically occurs by mutation of a gene that encodes for a protein such as an enzyme used in viral replication, and most typically in the case of HIV, reverse transcriptase, protease, or DNA polymerase, and in the case of HBV, DNA polymerase, or in the case of HCV, RNA polymerase, protease, or helicase. Recently, it has been demonstrated that the efficacy of a drug against HIV infection can be prolonged, augmented, or restored by administering the compound in combination or alternation with a second, and perhaps third, antiviral compound that induces a different mutation from that caused by the principle drug. The compounds can be used for combination are selected from the group consisting of a HBV polymerase inhibitor, interferon, TLR modulators such as TLR-7 agonists or TLR-9 agonists, therapeutic vaccines, immune activator of certain cellular viral RNA sensors, viral entry inhibitor, viral maturation inhibitor, distinct capsid assembly modulator, antiviral compounds of distinct or unknown mechanism, and combination thereof. Alternatively, the pharmacokinetics, biodistribution, or other parameter of the drug can be altered by such combination or alternation therapy. In general, combination therapy is typically preferred over alternation therapy because it induces multiple simultaneous stresses on the virus.

In certain embodiments, the method includes a regimen of controlling for cell death, by keeping the patient on anti-viral treatment such as tenofovir, etc. during delivery of the construct; then cycling the patient off of the anti-viral treatment for periods or time ranging from 2 weeks to 1 month, allowing for viral replication and limiting the viral cell death, so that there is not an overwhelming viral hepatic cell death situation, or similar cell death.

Following such a cycle, the patient is returned to anti-viral treatment, allowing for repair of the tissue; and repeating of the cycle every 8 weeks, every 12 weeks, every 16 weeks, every 20 weeks, etc. for as long as there is benefit to the patient.

Additional compounds for combination or alternation therapy for the treatment of HBV include 3TC, FTC, L-FMAU, interferon, adefovir dipivoxil, entecavir, telbivudine (L-dT), valtorcitabine (3′-valinyl L-dC), .beta.-D-dioxolanyl-guanine (DXG), .beta.-D-dioxolanyl-2,6-diaminopurine (DAPD), and .beta.-D-dioxolanyl-6-chloropurine (ACP), famciclovir, penciclovir, lobucavir, ganciclovir, and ribavirin.

### Kits

Additionally, certain components or embodiments of these recombinant nucleic acids, VLP products or pharmaceutical compositions thereof, can be provided in a kit. For example, any of the recombinant nucleic acids, or VLP products, can be provided frozen and packaged as a kit, alone or along with separate containers of any of the other agents from the pre-conditioning or post-conditioning steps, and optional instructions for use.

Some embodiments are also directed to any of the aforementioned compositions in a kit. In some embodiments, the kit may comprise ampoules, disposable syringes, capsules, vials, tubes, or the like. In some embodiments, the kit may comprise a single dose container or multiple dose containers comprising the topical formulation of embodiments herein. In some embodiments, each dose container may contain one or more unit doses. In some embodiments, the kit may include an applicator. In some embodiments, the kits include all components needed for the stages of conditioning/treatment. In some embodiments, the cellular compositions may have preservatives or be preservative-free (for example, in a single-use container). In some embodiments, the recombinant nucleic acids or VLP products may be prepared and frozen at a desired stage, suitable for shipping to a hospital or treatment center.

Additionally, in certain patients, it is expected that any of the methods or treatment regimens would be repeated periodically to boost the immune system response to the viral agent/s. Such periodic treatment can vary from once every week, month, to once every two months, to once every 3 months, to once every 4 months, to once every 5 months, to once every 6 months, or once every 7 months, or once every 8 months, or once every 9 months, or once every 10 months, or every 11 months, or once annually as a maintenance treatment for as long as the patient requires.

Although the invention has been described with respect to various preferred embodiments, it is not intended to be limited thereto, but rather those skilled in the art will recognize that variations and modifications may be made therein which are within the spirit of the invention and the scope of the appended claims.

In certain embodiments, test constructs described herein utilize AAV8 as the delivery vector because it targets liver cells; non-specific targeting will be tested in the various controls as indicated. TBG (Thyroxine Binding Globulin) is utilized as a liver-specific promoter, it is turned on by specific transcription factors made only in liver cells. This will drive expression of the recombinant transcript, which is in reverse complementary structure, and hence deemed non-functional and further undergoes degradation, without the presence of the viral transcription recognition signal, which in this case is the epsilon sequence (SEQ ID NO:1).

These experiments are designed to show that the test constructs will only be expressed in HBV-infected liver cells, where the HBV DNA polymerase is present, and interacts with the embedded HBV Epsilon transcription recognition sequences and transcribes the HBV “viral suicide” transcript. After this process, the sense HBV transcripts (which is the sense transcript recognized by the epsilon sequence, encoding Casp9) can now be transcribed and translated by host cell machinery to encode for Casp 9, which is under the strong constitutively active EFS promoter, that initiates the programmed cell death—apoptosis of the virally infected host cells, sparing any other uninfected cell from apoptosis.

These results are illustrated in the schematics, maps, graphs and tables from FIGS. 1-12, and are more fully explained below in Examples 1-6.

### Example 1

The HepAD38 is a cell line that replicates human hepatitis B virus (HBV) under conditions that can be regulated with tetracycline. In the presence of the antibiotic, this cell line is free of virus due to the repression of pregenomic (pg) RNA synthesis. Upon removal of tetracycline from the culture medium, the cells express viral pg RNA, accumulate subviral particles in the cytoplasm that contain DNA intermediates characteristic of viral replication, and secrete virus-like particles into the supernatant. Since the HepAD38 cell line can produce high levels of HBV DNA, it should be useful for analyses of the viral replication cycle that depend upon viral DNA synthesis in a synchronized fashion. In addition, this cell line has been formatted into a high-throughput, cell-based assay that permits the large-scale screening of diverse compound libraries for new classes of inhibitors of HBV replication. See, Ladner S. K. et al. Antimicrob Agents Chemother. 1997 August; 41(8):1715-20. Thus, the HepAD38 cell line a suitable in vitro model system for the study of human hepatitis B virus (HBV).

Hep G2 is an immortal cell line which was derived from the liver tissue of a 15-year-old American adolescent boy of European ancestry with a well-differentiated hepatocellular carcinoma. These cells are epithelial in morphology, have a modal chromosome number of 55, and are not tumorigenic in nude mice. The cells secrete a variety of major plasma proteins, e.g., albumin, and the acute-phase proteins fibrinogen, alpha 2-macroglobulin, alpha 1-antitrypsin, transferrin and plasminogen. They have been grown successfully in large-scale cultivation systems. Hepatitis B virus surface antigens have not been detected on these cells. Hep G2 will respond to stimulation with human growth hormone. Thus, Hep G2 cells are a suitable in vitro model system for the study of polarized human hepatocytes.

For testing the “viral specific cytotoxic” constructs described herein, utilizing HepG2 cells served as a control for normal, healthy liver cells. Since HepG2 cells are liver cells that are not infected with HBV, they should be unaffected by the test replication incompetent: AAV8-HBV-DRS1 (EF1a>HBV-rcCasp9), used in Experiment 1, with results shown in FIG. 2A and FIG. 2C.

In contrast, the HepAD38 cell line was utilized as the HBV+ model for testing one of the “viral specific cytotoxic” constructs described herein: the replication incompetent construct: AAV8-HBV-DRS1 (EF1a>HBV-rcCasp9) (vector shown in FIG. 2A). The results shown for Experiment 1, Groups 1-3 illustrate that the Group 2 test construct AAV8-HBV-DRS1 (EF1a>HBV-rcCasp9) in HepAD38 cells, exhibited viral specific cell death, as shown in the graphic data of FIG. 2B, and corresponding cell numbers and viability described in FIG. 2C.

### Example 2

Additionally, in Experiment 2, shown in FIG. 3A and FIG. 3B, and FIG. 4A, FIG. 4B and FIG. 4C, the AAV8-HBV-DRS1 (EF1a>HBV-rcCasp9) construct was further tested in HepG2 cells and HepAD38 cells and in certain test groups the Caspase-9 inhibitor Z-LEHD-FMK was used to illustrate that the killing was the result of Casp9 expression (See Group 3 and Group 5). The results from these experiments illustrate the viral specific cell death in the Group 2 HepAD38 cells, which exhibited an almost 80% rate of cell death. FIG. 5A is a vector map and FIG. 5B is a combined graph showing the effects of the test constructs and various controls in the HepG2 cells and HepAD38 cells to highlight the drastic cell death in the HepAD38 cells infected with the AAV8-HBV-DRS1 (EF1a>HBV-rcCasp9) construct.

### Example 3

Finally, in Experiment 3, the AAV8-HBV-DRS2 (TBG>HBV-rcCasp9) construct was tested. This construct contains a liver-specific promoter, thyroxine binding globulin (TBG). The results from these experiments illustrate the viral specific cell death in the Group 2 HepAD38 cells, which exhibited an almost 80% rate of cell death (FIG. 6A and FIG. 6B). In this grouping, the Group 1 cells were untransduced, the Group 2 cells were the test cells transduced with AAV8-HBV-DRS2 (TBG>HBV-rcCasp9); the Group 3 cells were the same with the addition of the Casp9 inhibitor z-LEHD.fmk; the Group 4 cells were contacted with only the Casp9 inhibitor z-LEHD.fmk as a control; the Group 5 cells were transduced with a construct containing GFP to further track the construct expression; the Group 6 cells were transduced with the same GFP construct and also incubated with the Casp9 inhibitor z-LEHD.fmk.

The test vector+Casp9 Inhibitor control groups in Experiments 2-3 illustrated that the cell death is caspase-9 initiated, and not from vector/DNA toxicity.

The GFP-AAV control groups in Experiments 2-3 illustrated that the cell death was not due to AAV toxicity.

### Example 4

In experiment 4, HBV-infected or HBV producing cells were treated with HBV RNA as provided herein, and mean percent cell death was calculated based on cell viability. By day 4, mean cell death in a variety of HBV-producing cells was 92% (ranging 88.6% to 95.8%). No significant cell death was observed in uninfected cells. RT and pan-caspase inhibitors individually prevented cell death in AAV-treated infected cells. These results show that HBV RNA construct selectively kills HBV-infected or producing cells.

### Example 5

### In Vivo Mouse Hepatitis Model Experiments

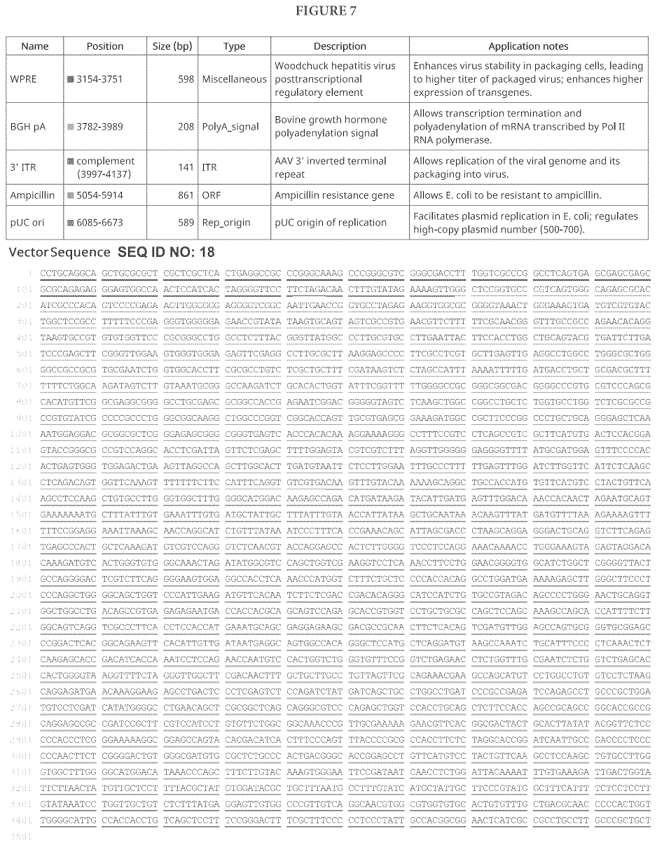

These constructs were further tested in an in vivo mouse model, and the results illustrate the viral specific cell death for the test constructs AAV8-HBV-DRS1 (EF1a>HBV-rcCasp9) and AAV8-HBV-DRS2 (TBG>HBV-rcCasp9) (FIG. 9).

In the above section, results from a novel in vitro proof-of-concept to co-opt the Hepatitis B Virus (HBV) polymerase (pol) to induce apoptosis of infected hepatocytes have been shown. An HBV transgenic mouse model was also used to evaluate in vivo the efficacy and specificity of the mechanism of these constructs and approach.

### Methods

AAV particles were packaged with a proprietary vector construct that expresses a non-functional non-coding (nc)RNA flanked between sequences specific to the reverse transcriptase (RT) domain of HBV pol (HBV pol/RT). The ncRNA would be recognized by HBV pol/RT and get reversely transcribed into double-stranded (ds)DNA which is encoded to overexpress caspase-9 (casp-9) (FIG. 1A and FIG. 1B). HBV transgenic mice, five per group, were injected with AAV particles expressing vectors under EF1a or liver-specific thyroxine binding globulin (TBG) promoter, a GFP vector or placebo. Peripheral blood was monitored weekly for liver enzymes and HBeAg. Heart, lungs, kidneys and liver were harvested on days 14 or 28 and evaluated for tissue distribution of AAV VP by IHC and Western Blot. Casp-9, caspase cleavage products and double staining of HBV core proteins with casp-9 were also evaluated by IHC. In addition, HBV pol-expressing or non-expressing HepG2-Red-Fluc cells were implanted into the liver of nude mice with matrigel to facilitate local tumor growth. Mice were then treated with AAVs expressing the test vector. At various time points, mice were injected with D-Luciferin substrate to quantify the changes in tumor size using the IVIS Lumina S5 Imaging System.

### Results

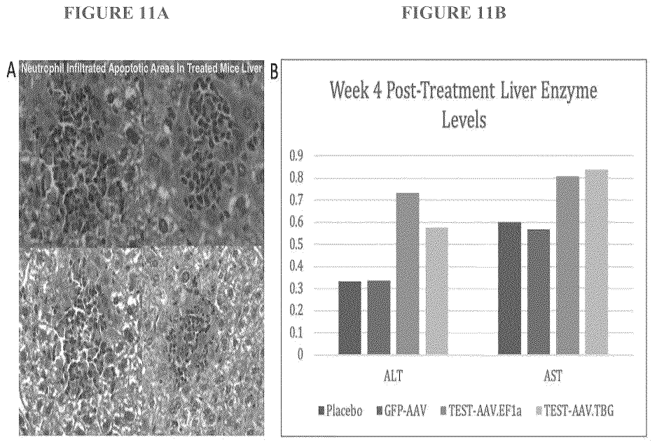

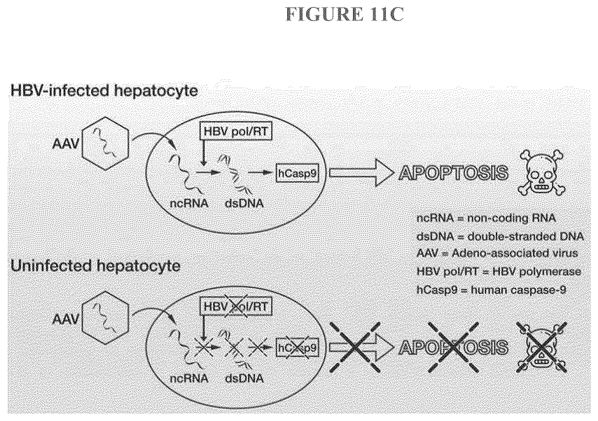

Organs harvested from transgenic mice on day 14 post-treatment showed significant casp-9 expression in liver and kidney cells expressing HBV but not in those not expressing HBV. Kidney tissues of mice treated with the vector carrying TBG promoter had no increased casp-9 expression compared to untreated and GFP controls. Moreover, cells in other organs that were stained positive for treatment AAV particles exhibited no casp-9 overexpression. Livers harvested on day 28 post-treatment showed diffuse apoptotic neutrophil-infiltrated areas in treatment groups but not in controls (FIG. 11A). At week 4 in treatment groups, ALT level increased 1.8-2.2-fold and AST levels increased 1.4-fold compared to GFP-AAV and placebo controls. (FIG. 11B). There were no significant changes in peripheral HBeAg. The results of the nude mice xenotransplantation cure study will be evaluated.

### Conclusions

A novel AAV vector to hijack HBV pol specifically induced overexpression of Casp-9 and apoptosis in HBV-expressing cells in vitro and in vivo. A schematic showing an overview of this process is shown in FIG. 11C. The lack of decrease in peripheral HBeAg was expected because regenerating liver tissue in transgenic mice constitutively express the antigen. These data suggest a potentially new pathway to treat or cure HBV infection.

SEQ ID NO: 1: HBV RNA Polymerase Epsilon Signal
UGUUCAUGUCCUACUGUUCAAGCCUCCAAGCUGUGCCUUGGGUGGCUUUGGGGCAUGGACA
SEQ ID NO: 2: EFS Promoter
GGCTCCGGTGCCCGTCAGTGGGCAGAGCGCACATCGCCCACAGTCCCCGAGAAGTTGGGGGGAGGGGTCGG
CAATTGATCCGGTGCCTAGAGAAGGTGGCGCGGGGTAAACTGGGAAAGTGATGTCGTGTACTGGCTCCGCC
TTTTTCCCGAGGGTGGGGGAGAACCGTATATAAGTGCAGTAGTCGCCGTGAACGTTCTTTTTCGCAACGGG
TTTGCCGCCAGAACACAGG
SEQ ID NO: 3: Caspase 9 (Casp9) Human ORF
ATGGACGAAGCGGATCGGCGGCTCCTGCGGCGGTGCCGGCTGCGGCTGGTGGAAGAGCTGCAGGTGGACCA
GCTCTGGGACGCCCTGCTGAGCCGCGAGCTGTTCAGGCCCCATATGATCGAGGACATCCAGCGGGCAGGCT
CTGGATCTCGGCGGGATCAGGCCAGGCAGCTGATCATAGATCTGGAGACTCGAGGGAGTCAGGCTCTTCCT
TTGTTCATCTCCTGCTTAGAGGACACAGGCCAGGACATGCTGGCTTCGTTTCTGCGAACTAACAGGCAAGC
AGCAAAGTTGTCGAAGCCAACCCTAGAAAACCTTACCCCAGTGGTGCTCAGACCAGAGATTCGCAAACCAG
AGGTTCTCAGACCGGAAACACCCAGACCAGTGGACATTGGTTCTGGAGGATTTGGTGATGTCGGTGCTCTT
GAGAGTTTGAGGGGAAATGCAGATTTGGCTTACATCCTGAGCATGGAGCCCTGTGGCCACTGCCTCATTAT
CAACAATGTGAACTTCTGCCGTGAGTCCGGGCTCCGCACCCGCACTGGCTCCAACATCGACTGTGAGAAGT
TGCGGCGTCGCTTCTCCTCGCTGCATTTCATGGTGGAGGTGAAGGGCGACCTGACTGCCAAGAAAATGGTG
CTGGCTTTGCTGGAGCTGGCGCAGCAGGACCACGGTGCTCTGGACTGCTGCGTGGTGGTCATTCTCTCTCA
CGGCTGTCAGGCCAGCCACCTGCAGTTCCCAGGGGCTGTCTACGGCACAGATGGATGCCCTGTGTCGGTCG
AGAAGATTGTGAACATCTTCAATGGGACCAGCTGCCCCAGCCTGGGAGGGAAGCCCAAGCTCTTTTTCATC
CAGGCCTGTGGTGGGGAGCAGAAAGACCATGGGTTTGAGGTGGCCTCCACTTCCCCTGAAGACGAGTCCCC
TGGCAGTAACCCCGAGCCAGATGCCACCCCGTTCCAGGAAGGTTTGAGGACCTTCGACCAGCTGGACGCCA
TATCTAGTTTGCCCACACCCAGTGACATCTTTGTGTCCTACTCTACTTTCCCAGGTTTTGTTTCCTGGAGG
GACCCCAAGAGTGGCTCCTGGTACGTTGAGACCCTGGACGACATCTTTGAGCAGTGGGCTCACTCTGAAGA
CCTGCAGTCCCTCCTGCTTAGGGTCGCTAATGCTGTTTCGGTGAAAGGGATTTATAAACAGATGCCTGGTT
GCTTTAATTTCCTCCGGAAAAAACTTTTCTTTAAAACATCATAA
SEQ ID NO: 4: Reverse Complement of Casp9
TTATGATGTTTTAAAGAAAAGTTTTTTCCGGAGGAAATTAAAGCAACCAGGCATCTGTTTATAAATCCCTT
TCACCGAAACAGCATTAGCGACCCTAAGCAGGAGGGACTGCAGGTCTTCAGAGTGAGCCCACTGCTCAAAG
ATGTCGTCCAGGGTCTCAACGTACCAGGAGCCACTCTTGGGGTCCCTCCAGGAAACAAAACCTGGGAAAGT
AGAGTAGGACACAAAGATGTCACTGGGTGTGGGCAAACTAGATATGGCGTCCAGCTGGTCGAAGGTCCTCA
AACCTTCCTGGAACGGGGTGGCATCTGGCTCGGGGTTACTGCCAGGGGACTCGTCTTCAGGGGAAGTGGAG
GCCACCTCAAACCCATGGTCTTTCTGCTCCCCACCACAGGCCTGGATGAAAAAGAGCTTGGGCTTCCCTCC
CAGGCTGGGGCAGCTGGTCCCATTGAAGATGTTCACAATCTTCTCGACCGACACAGGGCATCCATCTGTGC
CGTAGACAGCCCCTGGGAACTGCAGGTGGCTGGCCTGACAGCCGTGAGAGAGAATGACCACCACGCAGCAG
TCCAGAGCACCGTGGTCCTGCTGCGCCAGCTCCAGCAAAGCCAGCACCATTTTCTTGGCAGTCAGGTCGCC
CTTCACCTCCACCATGAAATGCAGCGAGGAGAAGCGACGCCGCAACTTCTCACAGTCGATGTTGGAGCCAG
TGCGGGTGCGGAGCCCGGACTCACGGCAGAAGTTCACATTGTTGATAATGAGGCAGTGGCCACAGGGCTCC
ATGCTCAGGATGTAAGCCAAATCTGCATTTCCCCTCAAACTCTCAAGAGCACCGACATCACCAAATCCTCC
AGAACCAATGTCCACTGGTCTGGGTGTTTCCGGTCTGAGAACCTCTGGTTTGCGAATCTCTGGTCTGAGCA
CCACTGGGGTAAGGTTTTCTAGGGTTGGCTTCGACAACTTTGCTGCTTGCCTGTTAGTTCGCAGAAACGAA
GCCAGCATGTCCTGGCCTGTGTCCTCTAAGCAGGAGATGAACAAAGGAAGAGCCTGACTCCCTCGAGTCTC
CAGATCTATGATCAGCTGCCTGGCCTGATCCCGCCGAGATCCAGAGCCTGCCCGCTGGATGTCCTCGATCA
TATGGGGCCTGAACAGCTCGCGGCTCAGCAGGGCGTCCCAGAGCTGGTCCACCTGCAGCTCTTCCACCAGC
CGCAGCCGGCACCGCCGCAGGAGCCGCCGATCCGCTTCGTCCAT
SEQ ID NO: 5: Reverse complement of EFS promoter
CCTGTGTTCTGGCGGCAAACCCGTTGCGAAAAAGAACGTTCACGGCGACTACTGCACTTATATACGGTTCT
CCCCCACCCTCGGGAAAAAGGCGGAGCCAGTACACGACATCACTTTCCCAGTTTACCCCGCGCCACCTTCT
CTAGGCACCGGATCAATTGCCGACCCCTCCCCCCAACTTCTCGGGGACTGTGGGCGATGTGCGCTCTGCCC
ACTGACGGGCACCGGAGCC
SEQ ID NO: 6: SV40 polyA
ACTTGTTTATTGCAGCTTATAATGGTTACAAATAAAGCAATAGCATCACAAATTTCACAAATAAAGCATTT
TTTTCACTGCATTCTAGTTGTGGTTTGTCCAAACTCATCAATGTATCTTATCATGTCTGGCTCT
SEQ ID NO: 7: Reverse complement of SV40 polyA
AGAGCCAGACATGATAAGATACATTGATGAGTTTGGACAAACCACAACTAGAATGCAGTGAAAAAAATGCT
TTATTTGTGAAATTTGTGATGCTATTGCTTTATTTGTAACCATTATAAGCTGCAATAAACAAGT
SEQ ID NO: 8: Construct (transcript): (1746 bp)
ATGTGTTCATGTCCTACTGTTCAAGCCTCCAAGCTGTGCCTTGGGTGGCTTTGGGGCATGGACAAGAGCCA
GACATGATAAGATACATTGATGAGTTTGGACAAACCACAACTAGAATGCAGTGAAAAAAATGCTTTATTTG
TGAAATTTGTGATGCTATTGCTTTATTTGTAACCATTATAAGCTGCAATAAACAAGTTTATGATGTTTTAA
AGAAAAGTTTTTTCCGGAGGAAATTAAAGCAACCAGGCATCTGTTTATAAATCCCTTTCACCGAAACAGCA
TTAGCGACCCTAAGCAGGAGGGACTGCAGGTCTTCAGAGTGAGCCCACTGCTCAAAGATGTCGTCCAGGGT
CTCAACGTACCAGGAGCCACTCTTGGGGTCCCTCCAGGAAACAAAACCTGGGAAAGTAGAGTAGGACACAA
AGATGTCACTGGGTGTGGGCAAACTAGATATGGCGTCCAGCTGGTCGAAGGTCCTCAAACCTTCCTGGAAC
GGGGTGGCATCTGGCTCGGGGTTACTGCCAGGGGACTCGTCTTCAGGGGAAGTGGAGGCCACCTCAAACCC
ATGGTCTTTCTGCTCCCCACCACAGGCCTGGATGAAAAAGAGCTTGGGCTTCCCTCCCAGGCTGGGGCAGC
TGGTCCCATTGAAGATGTTCACAATCTTCTCGACCGACACAGGGCATCCATCTGTGCCGTAGACAGCCCCT
GGGAACTGCAGGTGGCTGGCCTGACAGCCGTGAGAGAGAATGACCACCACGCAGCAGTCCAGAGCACCGTG
GTCCTGCTGCGCCAGCTCCAGCAAAGCCAGCACCATTTTCTTGGCAGTCAGGTCGCCCTTCACCTCCACCA
TGAAATGCAGCGAGGAGAAGCGACGCCGCAACTTCTCACAGTCGATGTTGGAGCCAGTGCGGGTGCGGAGC
CCGGACTCACGGCAGAAGTTCACATTGTTGATAATGAGGCAGTGGCCACAGGGCTCCATGCTCAGGATGTA
AGCCAAATCTGCATTTCCCCTCAAACTCTCAAGAGCACCGACATCACCAAATCCTCCAGAACCAATGTCCA
CTGGTCTGGGTGTTTCCGGTCTGAGAACCTCTGGTTTGCGAATCTCTGGTCTGAGCACCACTGGGGTAAGG
TTTTCTAGGGTTGGCTTCGACAACTTTGCTGCTTGCCTGTTAGTTCGCAGAAACGAAGCCAGCATGTCCTG
GCCTGTGTCCTCTAAGCAGGAGATGAACAAAGGAAGAGCCTGACTCCCTCGAGTCTCCAGATCTATGATCA
GCTGCCTGGCCTGATCCCGCCGAGATCCAGAGCCTGCCCGCTGGATGTCCTCGATCATATGGGGCCTGAAC
AGCTCGCGGCTCAGCAGGGCGTCCCAGAGCTGGTCCACCTGCAGCTCTTCCACCAGCCGCAGCCGGCACCG
CCGCAGGAGCCGCCGATCCGCTTCGTCCATCCTGTGTTCTGGCGGCAAACCCGTTGCGAAAAAGAACGTTC
ACGGCGACTACTGCACTTATATACGGTTCTCCCCCACCCTCGGGAAAAAGGCGGAGCCAGTACACGACATC
ACTTTCCCAGTTTACCCCGCGCCACCTTCTCTAGGCACCGGATCAATTGCCGACCCCTCCCCCCAACTTCT
CGGGGACTGTGGGCGATGTGCGCTCTGCCCACTGACGGGCACCGGAGCCTGTTCATGTCCTACTGTTCAAG
CCTCCAAGCTGTGCCTTGGGTGGCTTTGGGGCATGGACATAA
Reverse Complement sequence of “EFS promoter driving zsGreen gene”
flanked between HBV epsilon signals:
SEQ ID NO: 9
ATGTGTTCATGTCCTACTGTTCAAGCCTCCAAGCTGTGCCTTGGGTGGCTTTGGGGCATGGACAAGAGCCA
GACATGATAAGATACATTGATGAGTTTGGACAAACCACAACTAGAATGCAGTGAAAAAAATGCTTTATTTG
TGAAATTTGTGATGCTATTGCTTTATTTGTAACCATTATAAGCTGCAATAAACAAGTTTAGGGCAAGGCGG
AGCCGGAGGCGATGGCGTGCTCGGTCAGGTGCCACTTCTGGTTCTTGGCGTCGCTGCGGTCCTCGCGGGTC
AGCTTGTGCTGGATGAAGTGCCAGTCGGGCATCTTGCGGGGCACGGACTTGGCCTTGTACACGGTGTCGAA
CTGGCAGCGCAAGCGGCCACCGTCCTTCAGCAGCAGGTACATGCTCACGTCGCCCTTCAAGATGCCCTGCT
TGGGCACGGGGATGATCTTCTCGCAGGAGGGCTCCCAGTTGTCGGTCATCTTCTTCATCACGGGGCCGTCG
GCGGGGAAGTTCACGCCGTAGAACTTGGACTCGTGGTACATGCAGTTCTCCTCCACGCTCACGGTGATGTC
GGCGTTGCAGATGCACACGGCGCCGTCCTCGAACAGGAAGGAGCGGTCCCAGGTGTAGCCGGCGGGGCAGG
AGTTCTTGAAGTAGTCGACGATGTCCTGGGGGTACTCGGTGAACACGCGGTTGCCGTACATGAAGGCGGCG
GACAAGATGTCCTCGGCGAAGGGCAAGGGGCCGCCCTCCACCACGCACAGGTTGATGGCCTGCTTGCCCTT
GAAGGGGTAGCCGATGCCCTCGCCGGTGATCACGAACTTGTGGCCGTCCACGCAGCCCTCCATGCGGTACT
TCATGGTCATCTCCTTGGTCAGGCCGTGCTTGGACTGGGCCATCCTGTGTTCTGGCGGCAAACCCGTTGCG
AAAAAGAACGTTCACGGCGACTACTGCACTTATATACGGTTCTCCCCCACCCTCGGGAAAAAGGCGGAGCC
AGTACACGACATCACTTTCCCAGTTTACCCCGCGCCACCTTCTCTAGGCACCGGATCAATTGCCGACCCCT
CCCCCCAACTTCTCGGGGACTGTGGGCGATGTGCGCTCTGCCCACTGACGGGCACCGGAGCCTGTTCATGT
CCTACTGTTCAAGCCTCCAAGCTGTGCCTTGGGTGGCTTTGGGGCATGGACATAA

Reverse Complement Sequence of “EFS Promoter Driving zsGreen Gene” Flanked Between HBV Epsilon Signals:

ATGTGTTCATGTCCTACTGTTCAAGCCTCCAAGCTGTGCCTTGGGTGGCT
TTGGGGCATGGACAAGAGCCAGACATGATAAGATACATTGATGAGTTTG
GACAAACCACAACTAGAATGCAGTGAAAAAAATGCTTTATTTGTGAAAT
TTGTGATGCTATTGCTTTATTTGTAACCATTATAAGCTGCAATAAACAA
GTTTAGGGCAAGGCGGAGCCGGAGGCGATGGCGTGCTCGGTCAGGTGCC
ACTTCTGGTTCTTGGCGTCGCTGCGGTCCTCGCGGGTCAGCTTGTGCIG
GATGAAGTGCCAGTCGGGCATCTTGCGGGGCACGGACTTGGCCTTGTAC
ACGGTGTCGAACTGGCAGCGCAAGCGGCCACCGTCCTTCAGCAGCAGGT
ACATGCTCACGTCGCCCTTCAAGATGCCCTGCTTGGGCACGGGGATGAT
CTTCTCGCAGGAGGGCTCCCAGTTGTCGGTCATCTTCTTCATCACGGGG
CCGTCGGCGGGGAAGTTCACGCCGTAGAACTTGGACTCGTGGTACATGC
AGTTCTCCTCCACGCTCACGGTGATGTCGGCGTTGCAGATGCACACGGC
GCCGTCCTCGAACAGGAAGGAGCGGTCCCAGGTGTAGCCGGCGGGGCAG
GAGTTCTTGAAGTAGTCGACGATGTCCTGGGGGTACTCGGTGAACACGC
GGTTGCCGTACATGAAGGCGGCGGACAAGATGTCCTCGGCGAAGGGCAA
GGGGCCGCCCTCCACCACGCACAGGTTGATGGCCTGCTTGCCCTTGAAG
GGGTAGCCGATGCCCTCGCCGGTGATCACGAACTTGTGGCCGTCCACGC
AGCCCTCCATGCGGTACTTCATGGTCATCTCCTTGGTCAGGCCGTGCTT
GGACTGGGCCATCCTGTGTTCTGGCGGCAAACCCGTTGCGAAAAAGAAC
GTTCACGGCGACTACTGCACTTATATACGGTTCTCCCCCACCCTCGGGA
AAAAGGCGGAGCCAGTACACGACATCACTTTCCCAGTTTACCCCGCGCC
ACCTTCTCTAGGCACCGGATCAATTGCCGACCCCTCCCCCCAACTTCTC
GGGGACTGTGGGCGATGTGCGCTCTGCCCACTGACGGGCACCGGAGCCT
GTTCATGTCCTACTGTTCAAGCCTCCAAGCTGTGCCTTGGGTGGCTTTG
GGGCATGGACATAA

### Example 6

### Influenza-Specific Casp9 Construct and Experiments

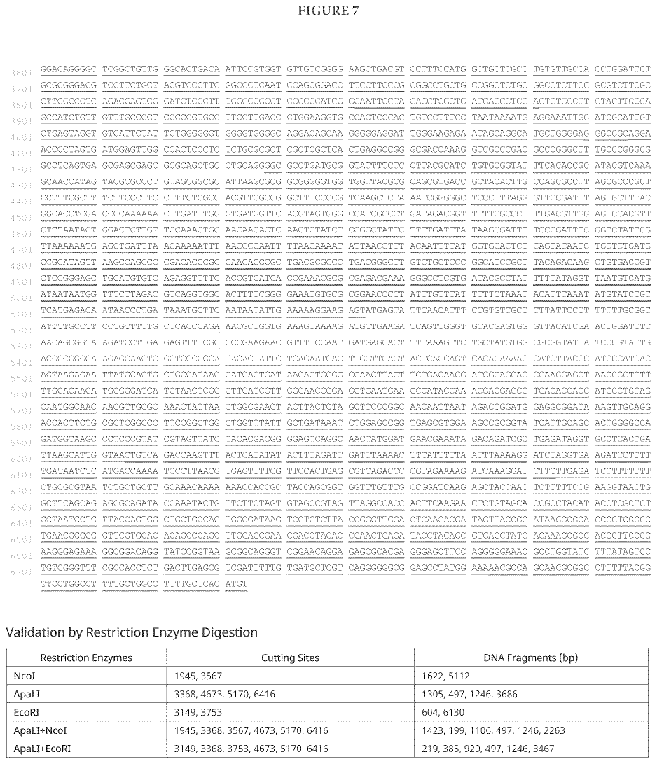

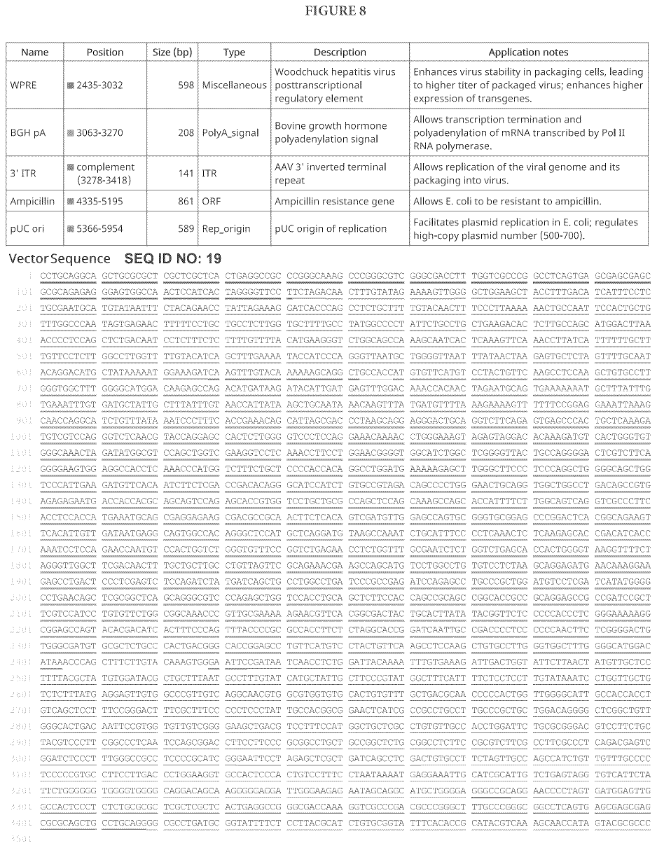

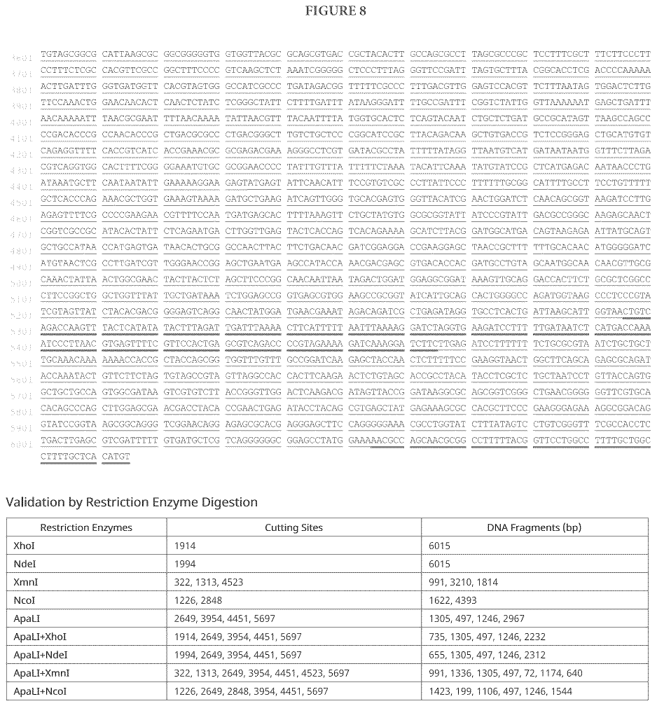

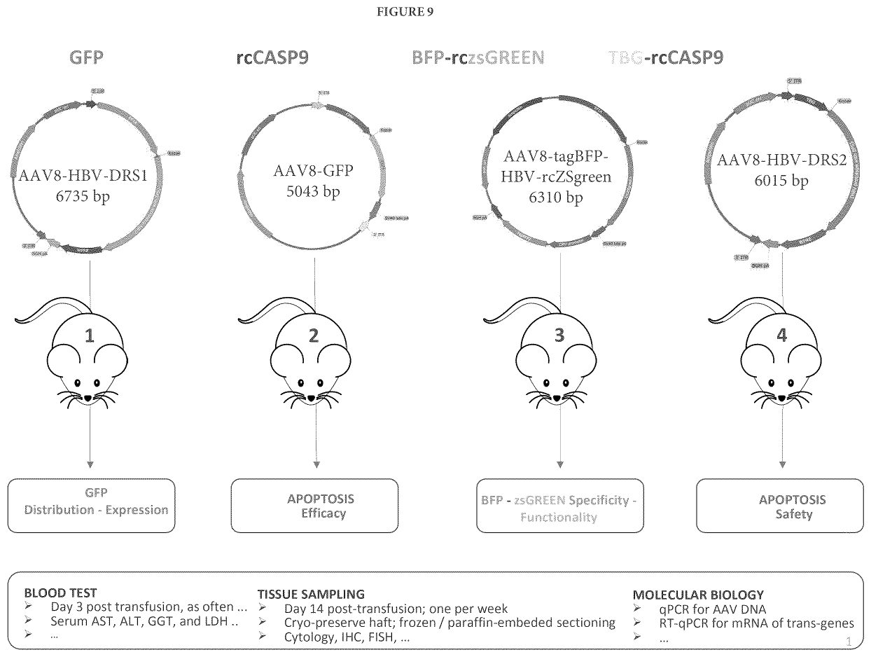

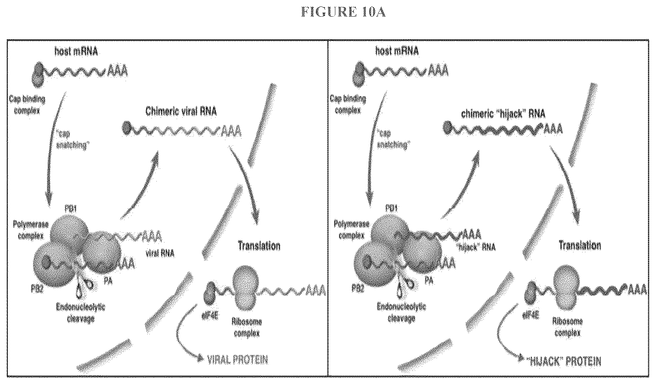

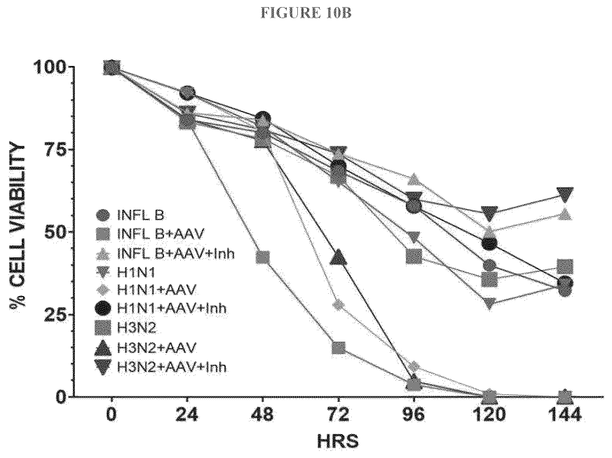

The following sequences and constructs will be utilized to test constructs designed to treat influenza viruses. Because influenza virus is a single-stranded negative-sense RNA virus that utilizes influenza polymerase complex to express viral proteins or replicate its viral genome, a vector construct expressing a negative-sense non-coding (nc)RNA has been designed to engage with influenza polymerase (in a similar manner to the ones designed specifically for HBV as described in Examples 1-4). The ncRNA “hijacks” the virus machinery to induce apoptosis specifically in infected cells, which is a potential anti-viral treatment (FIG. 10A and FIG. 10B).

### Methods

Adeno-associated virus (AAV) was packaged with a novel vector expressing ncRNA, (which can be thought of as influenza “hijack RNA”, or an RNA suicide vector for influenza infected cells), that transcribes the reverse complementary strand of zsGreen marker (AAV.infv.rcZsGreen) or caspase-9 (casp9) gene (AAV.infv.rcCasp9) between influenza genomic RNA non-coding sequence (NCS) regions that are highly conserved across several influenza virus strains. Embodiments of these sequences are shown below as SEQ ID NOs 10-15, 22-24. Madin-Darby Canine Kidney (MDCK) cells were infected with influenza A H1N1 or H3N2, or influenza B virus at 0.1 MOI, as an in vitro system to test these influenza (nc)RNA constructs. Influenza infected and uninfected MDCK cells were transduced with AAV.infv.rcZsGreen vector, and with AAV.infv.rcCasp9 in the presence and absence of casp9 inhibitor Z-LEHD-FMK to determine the functionality of the hijack vector, or RNA suicide vector for influenza infected cells. Flow cytometry was used to determine zsGreen expression. Cell viability and proliferation were evaluated daily by FACS, Annexin assay, and automated cell count.

### Results

All AAV.infv.rcZsGreen vector transduced influenza-infected cells successfully produced zsGreen protein, confirming that the hijack/suicide RNA constructs were recognized and transcribed by the polymerase complex in each influenza strain. Influenza infection killed 60%-68% of the untreated cells by day 6. 91%-95% of infected cells treated with casp9 hijack/suicide vector at 24 h post-influenza infection died on day 3 post-treatment. Casp-9 inhibition in the culture salvaged vector induced cell death and extended the lifespan of infected cells to 5-6 days (FIG. 10B). There was no significant cell death in uninfected control groups.

### Conclusions

A vector delivered in trans to engage with influenza polymerase hijacks the virus machinery to induce death in influenza infected cells 40% more rapidly than untreated infected cells. This effect was seen across viral strains, likely due to conserved nature of influenza polymerase. This novel approach could be used to develop an effective treatment for influenza.

Sequences
3′ Non-Coding Region of the vector
SEQ ID NO: 10
5′ CCTGCTTTTGCT 3′
5′ Non-Coding Region of the vector
SEQ ID NO: 11
5′ AGTAGAAACAAGG 3′
Influenza-specific Casp9 Vector
SEQ ID NO: 12
AGTAGAAACAAGGGTGTTTTTTATCATTATGATGTTTTAAAGAAAAGTTT
TTTCCGGAGGAAATTAAAGCAACCAGGCATCTGTTTATAAATCCCTTTC
ACCGAAACAGCATTAGCGACCCTAAGCAGGAGGGACTGCAGGTCTTCAG
AGTGAGCCCACTGCTCAAAGATGTCGTCCAGGGTCTCAACGTACCAGGA
GCCACTCTTGGGGTCCCTCCAGGAAACAAAACCTGGGAAAGTAGAGTAG
GACACAAAGATGTCACTGGGTGTGGGCAAACTAGATATGGCGTCCAGCT
GGTCGAAGGTCCTCAAACCTTCCTGGAACGGGGTGGCATCTGGCTCGGG
GTTACTGCCAGGGGACTCGTCTTCAGGGGAAGTGGAGGCCACCTCAAAC
CCATGGTCTTTCTGCTCCCCACCACAGGCCTGGATGAAAAAGAGCTTGG
GCTTCCCTCCCAGGCTGGGGCAGCTGGTCCCATTGAAGATGTTCACAAT
CTTCTCGACCGACACAGGGCATCCATCTGTGCCGTAGACAGCCCCTGGG
AACTGCAGGTGGCTGGCCTGACAGCCGTGAGAGAGAATGACCACCACGC
AGCAGTCCAGAGCACCGTGGTCCTGCTGCGCCAGCTCCAGCAAAGCCAG
CACCATTTTCTTGGCAGTCAGGTCGCCCTTCACCTCCACCATGAAATGC
AGCGAGGAGAAGCGACGCCGCAACTTCTCACAGTCGATGTTGGAGCCAG
TGCGGGTGCGGAGCCCGGACTCACGGCAGAAGTTCACATTGTTGATAAT
GAGGCAGTGGCCACAGGGCTCCATGCTCAGGATGTAAGCCAAATCTGCA
TTTCCCCTCAAACTCTCAAGAGCACCGACATCACCAAATCCTCCAGAAC
CAATGTCCACTGGTCTGGGTGTTTCCGGTCTGAGAACCTCTGGTTTGCG
AATCTCTGGTCTGAGCACCACTGGGGTAAGGTTTTCTAGGGTTGGCTTC
GACAACTTTGCTGCTTGCCTGTTAGTTCGCAGAAACGAAGCCAGCATGT
CCTGGCCTGTGTCCTCTAAGCAGGAGATGAACAAAGGAAGAGCCTGACT
CCCTCGAGTCTCCAGATCTATGATCAGCTGCCTGGCCTGATCCCGCCGA
GATCCAGAGCCTGCCCGCTGGATGTCCTCGATCATATGGGGCCTGAACA
GCTCGCGGCTCAGCAGGGCGTCCCAGAGCTGGTCCACCTGCAGCTCTTC
CACCAGCCGCAGCCGGCACCGCCGCAGGAGCCGCCGATCCGCTTCGTCC
ATTCCCCTGCTTTTGCT

### Key Features 5′:

SEQ ID NO:13: 5′ non-coding region of the vector: AGTAGAAACAAGG
Non-specific buffer sequence GTG
U₅₋₇ repeat that will be “stuttered” into PolyA signal during reverse transcription: TTTTTT
Non-specific buffer sequence, most common across influenza species: ATCA
Stop codon of the “final+sense Casp9 transcript”: TTA

### Key Features 3′:

Start codon of the “final+sense Casp9 transcript”: CAT
Non-specific buffer sequence: TCC
SEQ ID NO: 14: 3′ non-coding region of the vector: CCTGCTTTTGCT

### SEQ ID NO: 15

(−)ssRNA Transcript for Influenza Polymerase Complex Recognition and Reverse Transcription

### (Transcript Expressed by the Test Vector)

AGUAGAAACAAGGGUGUUUUUUAUCAUUAUGAUGUUUUAAAGAAAAGUUU
UUUCCGGAGGAAAUUAAAGCAACCAGGCAUCUGUUUAUAAAUCCCUUUC
ACCGAAACAGCAUUAGCGACCCUAAGCAGGAGGGACUGCAGGUCUUCAG
AGUGAGCCCACUGCUCAAAGAUGUCGUCCAGGGUCUCAACGUACCAGGA
GCCACUCUUGGGGUCCCUCCAGGAAACAAAACCUGGGAAAGUAGAGUAG
GACACAAAGAUGUCACUGGGUGUGGGCAAACUAGAUAUGGCGUCCAGCU
GGUCGAAGGUCCUCAAACCUUCCUGGAACGGGGUGGCAUCUGGCUCGGG
GUUACUGCCAGGGGACUCGUCUUCAGGGGAAGUGGAGGCCACCUCAAAC
CCAUGGUCUUUCUGCUCCCCACCACAGGCCUGGAUGAAAAAGAGCUUGG
GCUUCCCUCCCAGGCUGGGGCAGCUGGUCCCAUUGAAGAUGUUCACAAU
CUUCUCGACCGACACAGGGCAUCCAUCUGUGCCGUAGACAGCCCCUGGG
AACUGCAGGUGGCUGGCCUGACAGCCGUGAGAGAGAAUGACCACCACGC
AGCAGUCCAGAGCACCGUGGUCCUGCUGCGCCAGCUCCAGCAAAGCCAG
CACCAUUUUCUUGGCAGUCAGGUCGCCCUUCACCUCCACCAUGAAAUGC
AGCGAGGAGAAGCGACGCCGCAACUUCUCACAGUCGAUGUUGGAGCCAG
UGCGGGUGCGGAGCCCGGACUCACGGCAGAAGUUCACAUUGUUGAUAAU
GAGGCAGUGGCCACAGGGCUCCAUGCUCAGGAUGUAAGCCAAAUCUGCA
UUUCCCCUCAAACUCUCAAGAGCACCGACAUCACCAAAUCCUCCAGAAC
CAAUGUCCACUGGUCUGGGUGUUUCCGGUCUGAGAACCUCUGGUUUGCG
AAUCUCUGGUCUGAGCACCACUGGGGUAAGGUUUUCUAGGGUUGGCUUC
GACAACUUUGCUGCUUGCCUGUUAGUUCGCAGAAACGAAGCCAGCAUGU
CCUGGCCUGUGUCCUCUAAGCAGGAGAUGAACAAAGGAAGAGCCUGACU
CCCUCGAGUCUCCAGAUCUAUGAUCAGCUGCCUGGCCUGAUCCCGCCGA
GAUCCAGAGCCUGCCCGCUGGAUGUCCUCGAUCAUAUGGGGCCUGAACA
GCUCGCGGCUCAGCAGGGCGUCCCAGAGCUGGUCCACCUGCAGCUCUUC
CACCAGCCGCAGCCGGCACCGCCGCAGGAGCCGCCGAUCCGCUUCGUCC
AUUCCCCUGCUUUUGCU

### General Vector Structure

In some embodiments, the general vector structure has the following formula:

SEQ ID NO: 22-ORF-SEQ ID NO: 23,
wherein
SEQ ID NO: 22 is AGTAGAAACAAGGGTGTTTTTTATCATTA;
ORF is any protein coding sequence;
and
SEQ ID NO: 23 is CATTCCCCTGCTTTTGCT.

### SEQ ID NO:24

### GFP Marker Construct (in Place of Casp9) for Experimental Detection

AGTAGAAACAAGGGTGTTTTTTATCATTACTTGTACAGCTCGTCCATGC
CGAGAGTGATCCCGGCGGCGGTCACGAACTCCAGCAGGACCATGTGATC
GCGCTTCTCGTTGGGGTCTTTGCTCAGGGCGGACTGGGTGCTCAGGTAG
TGGTTGTCGGGCAGCAGCACGGGGCCGTCGCCGATGGGGGTGTTCTGCT
GGTAGTGGTCGGCGAGCTGCACGCTGCCGTCCTCGATGTTGTGGCGGAT
CTTGAAGTTCACCTTGATGCCGTTCTTCTGCTTGTCGGCCATGATATAG
ACGTTGTGGCTGTTGTAGTTGTACTCCAGCTTGTGCCCCAGGATGTTGC
CGTCCTCCTTGAAGTCGATGCCCTTCAGCTCGATGCGGTTCACCAGGGT
GTCGCCCTCGAACTTCACCTCGGCGCGGGTCTTGTAGTTGCCGTCGTCC
TTGAAGAAGATGGTGCGCTCCTGGACGTAGCCTTCGGGCATGGCGGACT
TGAAGAAGTCGTGCTGCTTCATGTGGTCGGGGTAGCGGCTGAAGCACTG
CACGCCGTAGGTCAGGGTGGTCACGAGGGTGGGCCAGGGCACGGGCAGC
TTGCCGGTGGTGCAGATGAACTTCAGGGTCAGCTTGCCGTAGGTGGCAT
CGCCCTCGCCCTCGCCGGACACGCTGAACTTGTGGCCGTTTACGTCGCC
GTCCAGCTCGACCAGGATGGGCACCACCCCGGTGAACAGCTCCTCGCCC
TTGCTCACCATTCCCCTGCTTTTGCT

### Example 7

In this experiment, Madin-Darby Canine Kidney (MDCK) cells were uninfected, infected with influenza A (H1N1 or H3N2) or influenza B virus. Next, the uninfected and infected cells were treated with AAV particles comprising hijack RNA coding for human casp9 or AAV particles comprising GFP. Three independent experiments, each in triplicates, were run.

Casp9 expression and its cleavage products (caspase 3 and 7) were measured via flow cytometry. Casp9 expression increased 4× with H1N1 influenza infection compared to uninfected cells. The hijack RNA AAV had no effect on uninfected cells, but when used on infected cells the “hijack” RNA AAV increased casp9 by 10× as compared to uninfected cells, and by 3× as compared to untreated infected cells.

The effectiveness of the hijack RNA AAV was measured daily by monitoring cell viability and proliferation with automated cell count and FACS analysis. Treatment of uninfected cells with the “hijack” RNA AAV produced no effect on cell viability. However, treatment of influenza B, H1N1, or H3N2 infected cells with the hijack RNA AAV increased cell viability by 120 hours as compared to untreated infected cells, or GFP AAV treated infected cells.

Infected cell percentage in the culture was monitored via influenza nucleoprotein intracellular staining using FACS analysis. Treatment with the hijack RNA AAV reduced percentage of H1N1, H3N2, or influenza B infected cells by 72 hours, and completely eradicated influenza infection by 96 hours.

These results show that the hijack RNA AAV that expresses caspase-9 effectively kills influenza infection in cell culture.

### Example 8

### Coronavirus Construct Design

Subgenomic (−)ssRNA synthesis from (+)ssRNA through CoV polymerase (RNA-dependent RNA-polymerase or RdRp). The coronaviral RdRp pauses at intergenic template-switching donor signals and is transferred by a copy-choice mechanism to a highly similar acceptor site near the 5′ end of the genome to copy the 5′-terminal leader. The transcription regulation sequences (TRS) function as template-switching signal. TRS is 5′ACGAAC3′ (SEQ ID NO: 16) on positive strand and 3′UGCUUG5′ (SEQ ID NO: 17) on negative strand. If the subgnomic mRNA (sgmRNA) still has multiple TRS in it, RdRp sees the new smRNA as a small genome, initiates negative strand synthesis on it, and switches template at an internal donor signal to make a negative strand template to synthesize a shorter internally nested sgmRNA. The leader RNA (60-80 nt) sequence always stays on the 3′ end of the subgenomic negative strand and 5′ end of the subgenomic mRNA.

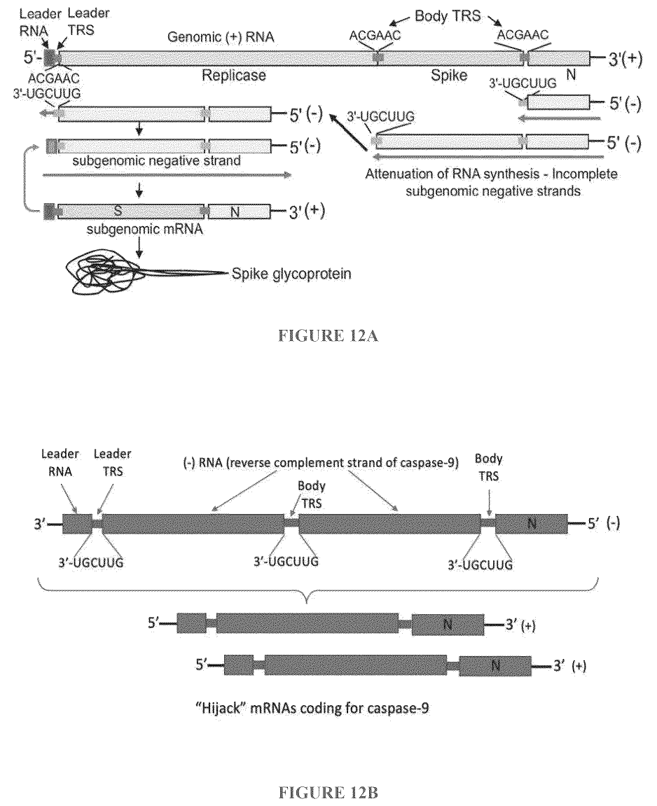

The subgenomic (−)ssRNA is an intermediate to produce new mRNAs for the viral protein synthesis. Thus, coronavirus “hijack RNAs” can be designed that are (−)ssRNA which mimic the leader RNA and TRS sequences but carry the negative strand of a gene of interest (e.g. caspase-9, for example). An overview of this strategy is shown in FIG. 12A and FIG. 12B.

### Standard Methods

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, NY; Sambrook and Russell (2001) Molecular Cloning, 3rd ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### Example 9

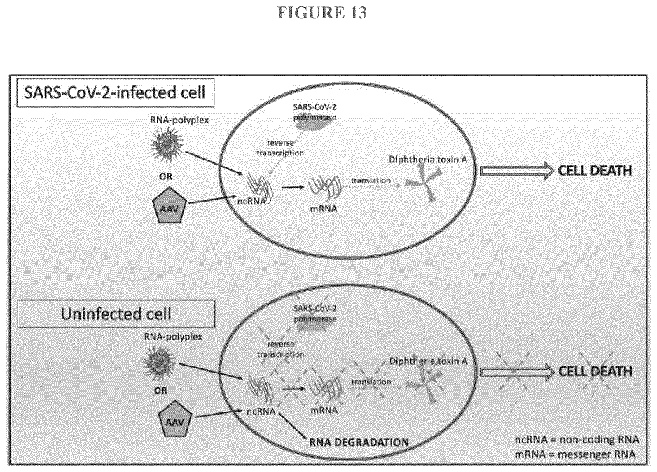

In this experiment, a synthetic RNA (“hijack RNA”) that was designed to be recognized by SARS-CoV-2 RNA-dependent-RNA-polymerase (RdRp) was tested for its ability to eradicate SARS-CoV-2 infection in cell culture. Upon recognition, hijack RNA would be transcribed into diphtheria toxin fragment A (DT-A), to induce apoptosis specifically in infected cells, which could be a potential treatment (FIG. 13).

### Methods

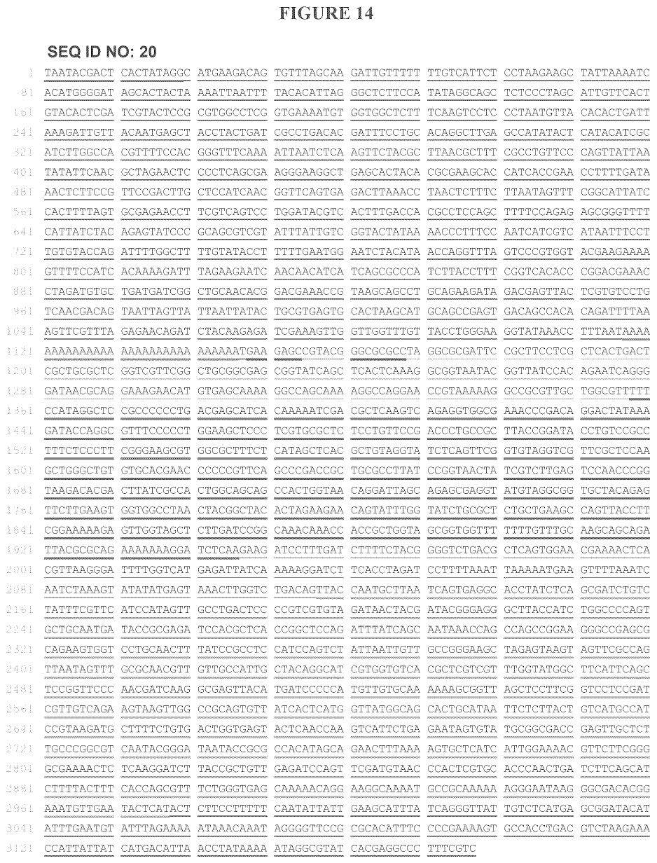

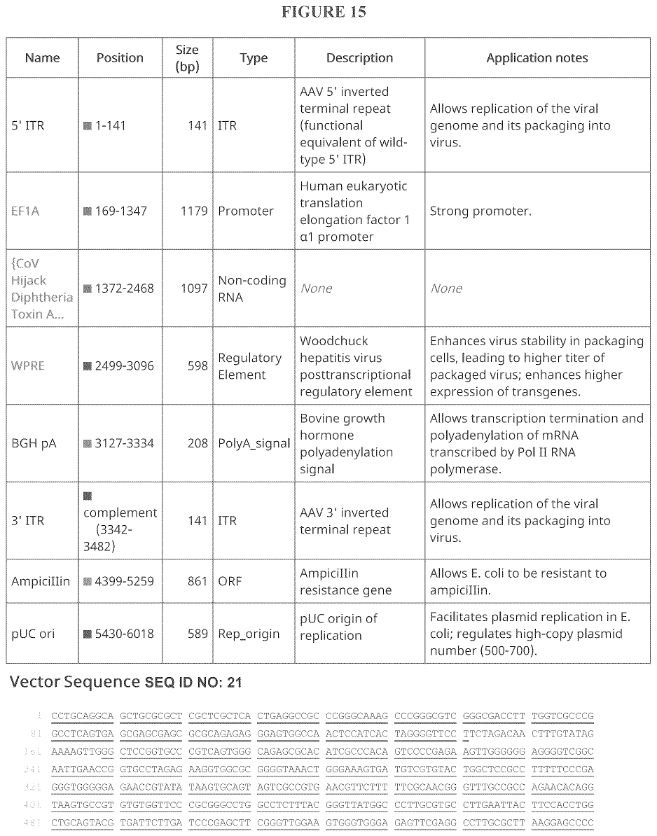

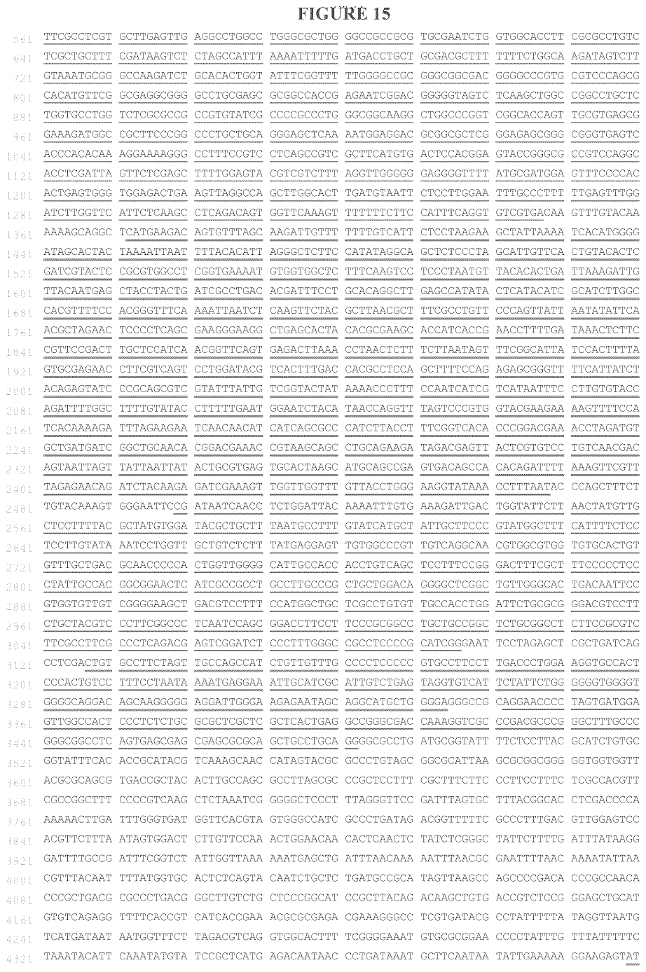

Adeno-associated virus (AAV) was packaged with a novel vector expressing SARS-CoV-2 hijack RNA (FIG. 15), which contains reverse complementary strand of DT-A cDNA and secondary structures of SARS-CoV-2 sgRNA. Vero cells were infected with SARS-CoV-2 USA-WA1/2020 strain at 0.1 MOI. SARS-CoV-2-infected and uninfected vero cells were transduced with test (FIG. 15) or control (GFP) AAVs. Uninfected jurkat, HEK and BHK-21 cells were also transduced with test AAV to further investigate potential off-target effects of hijack RNA in uninfected cells. Cell death and viability were evaluated daily by FACS and automated cell count. Eradication of infection in the cell cultures was established by the observation of cytopathic effects (CPE) via cell imaging and by FACS via intracellular staining of viral proteins. DT-A production in infected and uninfected cells was measured by western blot (WB) analysis of culture supernatants and cell lysates. Same experiments were repeated by transfecting the cells with in vitro transcribed hijack RNA (FIG. 14) at various (0-100 ng) concentrations. SARS-CoV-2 RdRp expressing stable vero and jurkat cell lines were also generated to assess RdRp-specific expression of the hijack RNA.

### Results

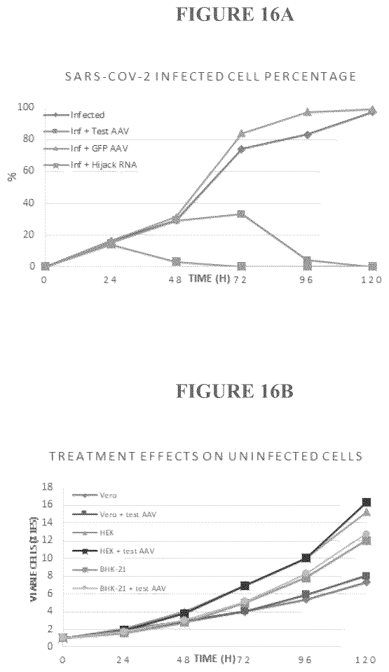

SARS-CoV-2 infection was successfully eradicated from the cultures within 48 h of test AAV transduction, confirmed by cell proliferation assays and the absence of CPE in cell imagery, as well as FACS analysis (FIG. 16A). Same results were observed within 24 h of hijack RNA transfection. Test AAV or hijack RNA had no effect on uninfected cells (FIG. 16B). Similar results were observed in RdRp expressing cell lines, confirming the hypothesized mechanism of action.

### CONCLUSION

An RNA delivered or expressed in trans to engage with SARS-CoV-2 RdRp hijacked the virus machinery and induced rapid death in infected cells but not in uninfected cells. Hijack RNA's translation into the kill molecule DT-A was dependent on viral RdRp as demonstrated in several different cell lines, confirming specificity and sensitivity of the treatment. This novel approach could be used to develop an effective treatment to eradicate COVID-19 infection.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1-52. (canceled)

53. A recombinant nucleic acid molecule comprising:
a negative strand nucleic acid molecule or a pgRNA nucleic acid molecule encoding a chemokine, a cytokine, an apoptosis inducing protein, diphtheria toxin A (or a fragment thereof), or any combination thereof flanked by a first and second viral transcription recognition signal;
a first promoter upstream (5′) of the first viral transcription recognition signal and
a second promoter adjacent and 5′ to the negative strand nucleic acid molecule or pgRNA nucleic acid molecule encoding a chemokine, a cytokine, an apoptosis inducing protein, diphtheria toxin A (or a fragment thereof), or any combination thereof.

54. The recombinant nucleic acid molecule of claim 53, wherein the negative strand nucleic acid molecule is negative sense RNA, negative sense DNA, single or double strand DNA that expresses a non-coding, negative sense RNA, or any combination thereof.

55. The recombinant nucleic acid molecule of claim 53, wherein the first and second viral transcription recognition signal is selected from a virus selected from a negative strand virus, an RNA reverse transcribing virus, or a DNA reverse transcribing virus.

56. The recombinant nucleic acid molecule of claim 53 further comprising a poly A tail downstream (3′) of the negative strand nucleic acid molecule or pgRNA nucleic acid molecule encoding a chemokine, a cytokine, an apoptosis inducing protein, or combination thereof.

57. A vector comprising the recombinant nucleic acid sequence of claim 53.

58. A pharmaceutical composition comprising any one of the recombinant nucleic acid sequence of claim 53.

59. A replication incompetent virus-like particle (VLP) comprising:
an optional translocation motif (TLM) fused to a capsid protein from a hepatitis B viral capsid or a hepatitis D viral capsid or coronavirus fusion protein;
a negative strand nucleic acid molecule or a pgRNA nucleic acid molecule encoding a chemokine, a cytokine, a apoptosis inducing protein, diphtheria toxin A (or a fragment thereof), or any combination thereof, flanked by a first and second viral transcription recognition signal;
a first promoter upstream (5′) of the first viral transcription recognition signal; and
a second promoter adjacent and 5′ to the negative strand nucleic acid molecule or pgRNA nucleic acid molecule encoding a chemokine, a cytokine, a apoptosis inducing protein, diphtheria toxin A (or a fragment thereof), or any combination thereof.

60. A replication incompetent virus-like particle (VLP) comprising:
a negative strand nucleic acid molecule or a pgRNA nucleic acid molecule encoding a chemokine, a cytokine, a apoptosis inducing protein, diphtheria toxin A (or a fragment thereof), or any combination thereof, flanked by a first and second target viral transcription recognition signal;
a first promoter upstream (5′) of the first viral transcription recognition signal; and
a second promoter adjacent and 5′ to the negative strand nucleic acid molecule or pgRNA nucleic acid molecule encoding a chemokine, a cytokine, a apoptosis inducing protein, diphtheria or toxin A (or fragment thereof), or any combination thereof, wherein the VLP exhibits tropism for the target virally infected cells.

61. A method of treating a viral infection in a subject in need thereof, the method comprising administering to the subject a recombinant nucleic acid molecule of claim 53, wherein the viral infection is:
an infection from a virus classified under Baltimore Classification IV, V, VI, or VII;
an infection from a virus selected from the group consisting of coronavirus, Hepatitis B virus, Hepatitis D virus, ebola virus, Marburg virus, human parainfluenza virus 1, measles virus, mumps virus, human respiratory syncytial virus, vesicular stomatitis Indiana virus, rabies virus, bovine ephemeral fever virus, lymphocytic choriomeningitis virus, Bunyamwera virus, Hantaan virus, Nairobi sheep disease virus, sandfly fever Sicilian virus, influenza virus A, influenza virus C, Thogoto virus, mouse mammary tumor virus, murine leukemia virus, avian leukosis virus, Mason-Pfizer monkey virus, bovine leukemia virus, human immunodeficiency virus 1, human spumavirus, duck hepatitis B virus, and a combination thereof; or
an infection from a virus selected from the group consisting of filovirus, paramyxovirus, morbillivirus, rubulavirus, pneumovirus, vesiculovirus, lyssavrius, ephemerovirus, arenavirus, bunyavirus, hantavirus, nairovirus, phlebovirus, Orthohepadnavirus, Avihepadnavirus, Mammalian type B retroviruses, Mammalian type C retroviruses, Avian type C retroviruses, Type D retroviruses, BLV-HTLV retroviruses, Lentivirus, Spumavirus, and a combination thereof.

62. A method of inducing an apoptotic response against a cell infected with a virus, in a subject in need thereof, the method comprising contacting the subject with a pharmaceutical composition comprising the recombinant nucleic acid molecule of claim 53.

63. A method of treating a hepatitis B, influenza, or coronavirus infection in a subject, the method comprising administering to the subject a pharmaceutical composition comprising the recombinant nucleic acid molecule of claim 53.
